SEQUENCE LISTING (9 sequences)

SEQ ID NO: 1 - DNA, 2747 bp, Homo sapiens
tgacgactcctgagttgcgcccatgctcttgtcagcttcgttttaggcgtagcatggccaggcagaagaaaatggggcaaagcgtgctccgggcggtcttctttttagtcctggggcttttgggtcattctcacggaggattccccaacaccatcagcataggtggacttttcatgagaaacacagtgcaggagcacagcgctttccgctttgccgtgcagttatacaacaccaaccagaacaccaccgagaagcccttccatttgaattaccacgtagatcacttggattcctccaatagtttttccgtgacaaatgctttctgctcccagttctcgagaggggtgtatgccatctttggattctatgaccagatgtcaatgaacaccctgacctccttctgtggggccctgcacacatcctttgttacgcctagcttccccactgacgcagatgtgcagtttgtcatccagatgcgcccagccttgaagggcgctattctgagtcttctgggtcattacaagtgggagaagtttgtgtacctctatgacacagaacgaggattttccatcctccaagcgattatggaagcagcagtgcaaaacaactggcaagtaacagcaaggtctgtgggaaacataaaggacgtccaagaattcaggcgcatcattgaagaaatggacaggaggcaggaaaagcgatacttgattgactgcgaagtcgaaaggattaacacaattttggaacaggttgtgatcctagggaaacactcaagaggttatcactacatgctcgctaacctgggttttactgatattttactggaaagagtcatgcatgggggagccaacattacaggtttccagattgtcaacaatgaaaaccctatggttcagcagttcatacagcgctgggtgaggctggatgaaagggaattccctgaagccaagaatgcaccactaaagtatacatctgcattgacacacgacgcaatactggtcatagcagaagctttccgctacctgaggaggcagcgagtagatgtgtcccggagaggaagtgctggagactgcttagcaaatcctgctgtgccctggagtcaaggaattgatattgagagagctctgaaaatggtgcaagtacaaggaatgactggaaatattcaatttgacacttatggacgtaggacaaattataccatcgatgtgtatgaaatgaaagtcagtggctctcgaaaagctggctactggaacgagtatgaaaggtttgtgcctttctcagatcagcaaatcagcaatgacagtgcatcctcagagaatcggaccatagtagtgactaccattctggaatcaccatatgtaatgtacaagaagaaccatgagcaactggaaggaaatgaacgatatgaaggctattgtgtagacctagcctatgaaatagccaaacatgtaaggatcaaatacaaattgtccatcgttggtgacgggaaatatggtgcaagggatccagagactaaaatatggaacggcatggttggggaacttgtctatgggagagctgatatagctgttgctccactcactataacattggtccgtgaagaagtcatagatttttcaaagccattaatgagcctgggcatctccatcatgataaagaagcctcagaaatcaaaaccaggcgtattctcatttctggatcccctggcttatgaaatctggatgtgcattgtctttgcttacattggagtcagcgtagttcttttcctagtcagcaggttcagtccttatgaatggcacttggaagacaacaatgaagaacctcgtgacccacaaagtcctcctgatcctccaaatgaatttggaatatttaacagtctttggttttccttgggtgcctttatgcagcaaggatgtgatatttctccaagatcactctccgggcgcattgttggaggggtttggtggttcttcaccctgatcataatttcttcctatactgccaatctcgctgctttcctgactgtggagaggatggtttctcccatagagagtgctgaagacttagctaaacagactgaaattgcatatgggaccctggactccggttcaacaaaagaatttttcagaagatccaaaattgctgtgtacgagaaaatgtggtcttacatgaaatcagcggagccatctgtgtttaccaaaacaacagcagacggagtggcccgagtgcgaaagtccaagggaaagttcgccttcctgctggagtcaaccatgaatgagtacattgagcagagaaaaccatgtgatacgatgaaagttggtggaaatctggattccaaaggctatggtgtggcaacccctaaaggctcagcattaggaaatgctgttaacctggcagtattaaaactgaatgagcaaggcctcttggacaaattgaaaaacaaatggtggtacgacaaaggagagtgcggcagcgggggcggtgactccaaggacaagaccagcgctctgagcctgagcaatgtggcaggcgttttctatatacttgtcggaggtctggggctggccatgatggtggctttgatagaattctgttacaaatcacgggcagagtccaaacgcatgaaactcacaaagaacacccaaaactttaagcctgctcctgccaccaacactcagaattatgctacatacagagaaggctacaacgtgtatggaacagagagtgttaagatctagggatccctt

SEQ ID NO: 2 - DNA, 3056 bp, Homo sapiens
tgacgactcctgagttgcgcccatgctcttgtcagcttcgttttaggcgtagcatggccaggcagaagaaaatggggcaaagcgtgctccgggcggtcttctttttagtcctggggcttttgggtcattctcacggaggattccccaacaccatcagcataggtggacttttcatgagaaacacagtgcaggagcacagcgctttccgctttgccgtgcagttatacaacaccaaccagaacaccaccgagaagcccttccatttgaattaccacgtagatcacttggattcctccaatagtttttccgtgacaaatgctttctgctcccagttctcgagaggggtgtatgccatctttggattctatgaccagatgtcaatgaacaccctgacctccttctgtggggccctgcacacatcctttgttacgcctagcttccccactgacgcagatgtgcagtttgtcatccagatgcgcccagccttgaagggcgctattctgagtcttctgggtcattacaagtgggagaagtttgtgtacctctatgacacagaacgaggattttccatcctccaagcgattatggaagcagcagtgcaaaacaactggcaagtaacagcaaggtctgtgggaaacataaaggacgtccaagaattcaggcgcatcattgaagaaatggacaggaggcaggaaaagcgatacttgattgactgcgaagtcgaaaggattaacacaattttggaacaggttgtgatcctagggaaacactcaagaggttatcactacatgctcgctaacctgggttttactgatattttactggaaagagtcatgcatgggggagccaacattacaggtttccagattgtcaacaatgaaaaccctatggttcagcagttcatacagcgctgggtgaggctggatgaaagggaattccctgaagccaagaatgcaccactaaagtatacatctgcattgacacacgacgcaatactggtcatagcagaagctttccgctacctgaggaggcagcgagtagatgtgtcccggagaggaagtgctggagactgcttagcaaatcctgctgtgccctggagtcaaggaattgatattgagagagctctgaaaatggtgcaagtacaaggaatgactggaaatattcaatttgacacttatggacgtaggacaaattataccatcgatgtgtatgaaatgaaagtcagtggctctcgaaaagctggctactggaacgagtatgaaaggtttgtgcctttctcagatcagcaaatcagcaatgacagtgcatcctcagagaatcggaccatagtagtgactaccattctggaatcaccatatgtaatgtacaagaagaaccatgagcaactggaaggaaatgaacgatatgaaggctattgtgtagacctagcctatgaaatagccaaacatgtaaggatcaaatacaaattgtccatcgttggtgacgggaaatatggtgcaagggatccagagactaaaatatggaacggcatggttggggaacttgtctatgggagagctgatatagctgttgctccactcactataacattggtccgtgaagaagtcatagatttttcaaagccattaatgagcctgggcatctccatcatgataaagaagcctcagaaatcaaaaccaggcgtattctcatttctggatcccctggcttatgaaatctggatgtgcattgtctttgcttacattggagtcagcgtagttcttttcctagtcagcaggttcagtccttatgaatggcacttggaagacaacaatgaagaacctcgtgacccacaaagtcctcctgatcctccaaatgaatttggaatatttaacagtctttggttttccttgggtgcctttatgcagcaaggatgtgatatttctccaagatcactctccgggcgcattgttggaggggtttggtggttcttcaccctgatcataatttcttcctatactgccaatctcgctgctttcctgactgtggagaggatggtttctcccatagagagtgctgaagacttagctaaacagactgaaattgcatatgggaccctggactccggttcaacaaaagaatttttcagaagatccaaaattgctgtgtacgagaaaatgtggtcttacatgaaatcagcggagccatctgtgtttaccaaaacaacagcagacggagtggcccgagtgcgaaagtccaagggaaagttcgccttcctgctggagtcaaccatgaatgagtacattgagcagagaaaaccatgtgatacgatgaaagttggtggaaatctggattccaaaggctatggtgtggcaacccctaaaggctcagcattaggaacgcctgtaaaccttgcagtattgaaactcagtgaacaaggcatcttagacaagctgaaaaacaaatggtggtacgataagggggaatgtggagccaaggactccgggagtaaggacaagaccagcgctctgagcctgagcaatgtggcaggcgttttctatatacttgtcggaggtctggggctggccatgatggtggctttgatagaattctgttacaaatcacgggcagagtccaaacgcatgaaactcacaaagaacacccaaaactttaagcctgctcctgccaccaacactcagaattatgctacatacagagaaggctacaacgtgtatggaacagagagtgttaagatctagggatcccttcccactggaggcatgtgatgagaggaaatcaccgaaaacgtggctgcttcaaggatcctgagccagatttcactctccttggtgtcgggcatgacacgaatattgctgatggtgcaatgacctttcaataggaaaaactgattttttttttccttcagtgccttatggaacactctgagactcgcgacaatgcaaaccatcattgaaatctttttgctttgcttgaaaaaaaataattaaaataaaaaccaacaaaaatggacatgcatcaaacccttgatgtattaatatttattatagttttcatta

SEQ ID NO: 3 - RNA, 14 nt, Artificial sequence
  Consensus sequence for the hairpin ribozyme
nnnbngucnnnnnn

SEQ ID NO: 4 - DNA, 21 bp, Artificial sequence
  synthetic single strand oligonucleotide
gcagtgggccttcaactctac

SEQ ID NO: 5 - DNA, 24 bp, Artificial sequence
  synthetic single strand oligonucleotide
ggatgggactctaattcgtatatc

SEQ ID NO: 6 - DNA, 21 bp, Artificial sequence
  synthetic single strand oligonucleotide
caggtccaggggtcttggtcc

SEQ ID NO: 7 - DNA, 22 bp, Artificial sequence
  synthetic single strand oligonucleotide
ggcagaccgagatgaatcctca

SEQ ID NO: 8 - DNA, 20 bp, Artificial sequence
  Synthetic single strand oligonucleotide
cgatacttgattgactgcga

SEQ ID NO: 9 - DNA, 20 bp, Artificial sequence
  Synthetic single strand oligonucleotide
tactatggtccgattctctg

(19) United States
(12) Patent Application Publication
(10) Pub. No.: US 2005/0214217 A1
(43) Pub. Date: Sep. 29, 2005

(54) Methods of and pharmaceutical compositions for modulating T-lymphocyte adhesion, migration, gene expression and function by Glutamate and analogs thereof

(76) Inventor: Mai Levite, Savyon (IL)

Correspondence Address: G.E. EHRLICH (1995) LTD.;c/o ANTHONY CASTORINA, SUITE 207, 2001 JEFFERSON DAVIS HIGHWAY, ARLINGTON, VA, 22202, US

(21) Appl. No.: 10/809,425

(22) Filed: Mar. 26, 2004

(51) Int. Cl. A61K 49/00; C12Q 1/00

(52) U.S. Cl. 424/9.1; 435/4

(57) ABSTRACT

Methods and compositions for the direct modulation of T-cell activity by the action of glutamate and glutamate functional analogs, effecting gene expression and cytokine secretion, integrin-mediated adhesion, and chemotactic migration for the treatment of infectious diseases, inhibition and prevention of tumor growth and dissemination, prevention and treatment of neurological disease, psychopathology, neuronal damage in CNS disease, infection and injury, containment of auto-immune and other injurious inflammatory processes, enhancement of anti-tumor immune surveillance and prevention of host rejection of engrafted tissue are disclosed.

### FIELD AND BACKGROUND OF THE INVENTION

The present invention relates to novel methods for the direct modulation of T-cell activity by the action of Glutamate and Glutamate functional analogs and, more particularly, to methods for the treatment of infectious diseases, inhibition and prevention of tumor growth and dissemination, prevention and treatment of neurological disease, psychopathology, neuronal damage in CNS disease, infection and injury, containment of auto-immune and other injurious inflammatory processes, enhancement of anti-tumor immune surveillance and prevention of host rejection of engrafted tissue. Specifically, the present invention employs GluR3 Glutamate receptor-mediated regulation of de novo gene expression, integrin activation and cytokine secretion, in turn effecting integrin-mediated adhesion, chemotactic migration and, ultimately, T-cell participation in inflammation and surveillance in infection, injury and disease.

T-cells in immunity and disease: Immune responses are largely mediated by a diverse collection of peripheral blood cells termed leukocytes. The leukocytes include lymphocytes, granulocytes and monocytes. Granulocytes are further subdivided into neutrophils, eosinophils and basophils. Lymphocytes are further subdivided into T and B lymphocytes. T-lymphocytes originate from lymphocytic-committed stem cells of the embryo. Differentiation occurs in the thymus and proceeds through prothymocyte, cortical thymocyte and medullary thymocyte intermediate stages, to produce various types of mature T-cells. These subtypes include CD4+ T cells (also known as T helper and T inducer cells), which, when activated, have the capacity to stimulate other immune system cell types. The T-helper cells are further subdivided into the Th1, Th2 and Th3 cells, primarily according to their specific cytokine secretion profile and function. T cells also include suppressor/regulator T cells (previously known as cytotoxic/suppressor T cells), which, when activated, have the capacity to lyse target cells and suppress CD4⁺ mediated effects.

T-cell activation: Immune system responses are elicited in a variety of situations. The most frequent response is as a desirable protection against infectious microorganisms. The current dogma is that in the organism, under physiological conditions, resting T-cells are activated and triggered to function primarily by antigens which bind to T-cell receptor (TCR) after being processed and presented by antigen-presenting cells, or by immunocyte-secreted factors such as chemokines and cytokines, operating through their own receptors. Experimentally, T-cells can be activated by various non-physiological agents such as phorbol esters, mitogens, ionomycin, and anti-CD3 antibodies. To identify novel physiological means directly activating and/or regulating T-cells in conditions of health and disease, especially in non-lymphoid environments (e.g. brain) and in a TCR-independent manner, remains a challenge of scientific and clinical importance.

In recent years, it has become evident that specific immune responses and diseases are associated with T-helper (Th) functions. Among these are anti-viral, anti-bacterial and anti-parasite immune responses, mucosal immune responses and systemic unresponsiveness (mucosally induced tolerance), autoimmune reactions and diseases, allergic responses, allograft rejection, graft-versus host disease and others. Furthermore, specific T-cell mediated proinflammatory functions may have either beneficial or detrimental effects on specific neoplasias: on the one hand, proinflammatory cytokines may assist in anti-tumor immune surveillance, and, on the other, elevated levels of proinflammatory cytokines were found within chronically inflamed tissues that show increased incidence of neoplasia.

In general, CD4+ T-cells can be divided into at least two major mutually exclusive subsets, Th1 and Th2, distinguished according to their cytokine secretion profile. Th1 cells secrete mainly IFN-γ, TNF-α and IL-2, their principal effector function being in phagocyte-mediated defense against infections. The Th1 cells are usually associated with inflammation, and induce cell-mediated responses.

Essential and beneficial immunity cannot take place without Th1 cytokines, but their over or dis-regulated production leads to numerous detrimental clinical consequences. Th2 cells induce B-cell proliferation and differentiation, and thus, induce immunoglobulin production. Cytokines from Th2 cells (mainly IL-4, IL-10 and IL-13) can also antagonize the effects of Th1 cell-mediated reactivities, inhibiting potentially injurious Th1 responses.

Modification of T-cell activity: Therapeutic application of T-cell modulating agents has been proposed for the treatment of conditions characterized by both immune deficiency and chronic inflammation. For example, U.S. Pat. No. 5,632,983 to Hadden discloses a composition consisting of peptides of thymus extract, and natural cytokines, for stimulation of cell mediated immunity in immune deficient conditions. Although significant enhancement of a number of cell mediated immune functions was demonstrated the effects were highly non-specific, as could be expected when employing poorly defined biologically derived materials.

Recently, Butcher et al. (U.S. Pat. No. 6,245,332) demonstrated the specific interaction of chemokine ligands TARC and MDC with the CCR4 receptors of memory T-cells, enhancing interaction of these cells with vascular epithelium and promoted T-cell extravasation. Therapeutic application of CCR4 agonists was disclosed for enhanced T-cell localization, and of antagonists for inhibition of immune reactivity, as an anti-inflammatory agent. Although the ligands were characterized, and identified in inflamed tissue, no actual therapeutic effects of agonists or antagonists were demonstrated.

Inhibition of a number of T-cell functions has been the target of many proposed anti-inflammatory therapies. Haynes et al. (U.S. Pat. No. 5,863,540) disclosed the use of anti-CD44 (cell adhesion molecule effecting T-cell activation) antibody for treatment of autoimmune conditions such as Rheumatoid Arthritis. Godfrey et al. (U.S. Pat. No. 6,277,962) disclosed a purified ACT-4 T-cell surface receptor expressed in activated CD4+ and CD8+ T-cells, and proposed the use of anti-ACT-4 antibodies to achieve downregulation of T-cell activation. Similarly, Weiner et al. (U.S. Pat. Nos. 6,077,509 and 6,036,457) proposed treatment with peptides containing immunodominant epitopes of myelin basic protein (associated with Multiple Sclerosis) for the specific supression of CD4+ T-cell activity in this central nervous system autoimmune condition. However, none of the proposed applications were able to demonstrate a specific, primary effect on T-cell activation mediated by defined surface components.

Neurotransmitters and Immune System Function: It is generally accepted that the immune, nervous and endocrine systems are functionally interconnected. The significance of direct neuronal signaling on immune system components, including T-cells, can be appreciated considering the extensive innervation of all primary and secondary lymphoid tissue; the presence of both peptidergic and non-peptidergic neurotransmitters in capillaries and at sites of inflammation, injury or infection; and the demonstrated expression of specific receptors for various neurotransmitters on T-cell (and other immune system components) surface membrane.

Specific modulation of immune function has been demonstrated for a number of neurotransmitters. Recently, neuropeptides somatostatin (SOM), calcitonin gene related peptide (CGRP), neuropeptide Y (NPY), substance P and also dopamine were found to interact directly with specific receptors on the T-cell surface, and to either activate or inhibit T-cell functions such as cytokine secretion, adhesion to extracellular components and apoptosis, depending on T-cell lineage and activation states (Levite, M.: Nerve Driven Immunity. The direct effects of neurotransmitters on T-cell function. Ann NY Acad. Sci.: 307-21). Similarly, in vitro application of physiological concentrations of the neurotransmitters SOM, Sub P, CGRP and NPY was found to directly induce both typical and non-typical cytokine and chemokine secretion from human Th1 and Th2 T-cells and from human intestinal epithelial cell lines, thus either blocking or evoking immune function (Levite, M. Nervous immunity: neurotransmitters, extracellular K⁺ and T-cell function. Trends Immunol. 2001 January; 22(1):2-5). Clearly, immune function is sensitive to neurogenic control.

A number of therapeutic applications of immune modulation by direct or indirect manipulation of neurotransmitter availability or function have been proposed. In one, botulinum toxin's peptide-lytic activity is employed to reduce the effect of immune-active neurotransmitters Sub P, CGRP, VIP, cytokines IL-1 and IL-6 and others, such as NK-1 on neurogenic inflammatory conditions such as arthritis, synovitis, migraine and asthma (U.S. Pat. No. 6,063,763 to First). Hitzig (U.S. Pat. No. 5,658,955) proposes the combined application of the neurotransmitters dopamine and serotonin for complex inhibition and stimulation of various immune functions, for the treatment of AIDS and HIV infection, cancers, migraine, autoimmune inflammatory and allergic conditions, chronic fatigue syndrome and fibromyalgia. On the whole, however, the immune modulation of these inventions is of a broad and non-specific nature, with significant likelihood of undesirable complications and side effects in practice.

Glutamate in the CNS: L-Glutamate mediates excitatory neurotransmission in the mammalian central nervous system through its action at Glutamate receptors. There are two broad classes of Glutamate receptors, known as the ionotropic Glutamate receptor and the metabotropic Glutamate receptor. Within the class of ionotropic Glutamate receptor are three groups, known as the N-methyl-D-aspartate (NMDA), (R,S)-2-amino-3-(3-hydroxy-5-methyl-isoxazol-4-yl)propanoate (AMPA) and kainate (KA) receptors. Molecular biological studies have established that AMPA receptors are composed of subunits (GluR1-4) that can assemble to form functional channels. Five kainate receptors, classified as either high affinity (KA1 and KA2) or low affinity (GluR5, GluR6 and GluR7) kainate receptors have been identified. (Bleakman et al, Molecular Pharmacology, 1996, Vol. 49, No. 4, pgs. 581-585).

Recently, AMPA receptors have been widely studied for their possible contributions to neurological pathologies such as stroke, trauma and epilepsy (Fisher and Bogousslavsky, J. Amer. Med. Assoc. 270: 360, 1993; Yamaguchi et al., Epilepsy Res. 15: 179, 1993). Several distinct subtypes of AMPA and kainate receptors have been cloned as well (see review by Nakanishi, Brain Res Brain Res Rev 1998, May; 26(2-3):230-35). Of particular relevance are the AMPA receptors designated GluR1, GluR2, GluR3, and GluR4 (also termed GluRA through GluRD), each of which exists in one of two forms, termed flip and flop, which arise by RNA alternative splicing. GluR1, GluR3 and GluR4, when expressed as homomeric and heteromeric receptors, are permeable to Ca⁺⁺, and are therefore examples of receptor-operated Ca⁺⁺ channels. Expression of GluR2 alone or in combination with the other subunits gives rise to a receptor which is largely impermeable to Ca⁺⁺. As most native AMPA receptors studied in situ are not Ca⁺⁺-permeable (discussed above), it is believed that such receptors in situ possess at lest one GluR2 subunit.

The activity of the AMPA receptor is regulated by a number of modulatory sites that can be targeted by selective antagonists (Honore et al., Science 241: 701, 1988; Donevan and Rogawski, Neuron 10: 51, 1993). Competitive antagonists such as NBQX act at the Glutamate binding site, whereas compounds such as GYKI 52466 appear to act noncompetitively at an associated allosteric site.

The effects of Glutamate on lymphocyte function have been investigated in respect to modulation of cell activation. Lombardi (Lombardi, et al, Br J Pharmacol 2001 July; 133(6)936-44) reported induction of Ca⁺⁺ influx and proliferation by Glutamate in PHA or mAb activated human peripheral lymphocytes, and inhibition of Glutamate potentiation by AMPA-specfic receptor antagonists NBQX and KYNA. Since no effect was observed with even high levels (1 mM) of Glutamate on unstimulated, resting lymphocytes, the authors concluded that human lymphocyte Glutamate ionotropic receptors cannot provide primary stimulation, but rather function as modulators of cell activation in conjunction with other, primary activators. In another study measuring NK lymphocyte responses, the blockage of NMDA Glutamate receptors and/or Glutamate release inhibited recall of the conditioned NK cell response, further supporting Glutamate's role in neuro-immune modulation (Kuo, et al 2001 Aug. 30; 118(2):245-55). However, no studies to date have demonstrated primary responsiveness of T-cells to Glutamate, or the presence of either ionotropic or metabotropic Glutamate receptor-mediated function specifically in T-cells.

CNS Excitotoxicity: It is well established that, upon hypoxia-ischemia of the immature or adult brain or upon head injury, Glutamate exerts an excessive excitation of neurons leading to their death. Excitatory Glutamate stimulation is also believed to play a major role in other neurodegenerative disorders such as amyotrophic lateral sclerosis, glaucoma, Alzheimer's disease and epilepsy.

When in excess, Glutamate, the major excitatory neurotransmitter in the central nervous system, is directly toxic to neuronal tissues. Glutamate neurotoxicity is mediated by the activation of Glutamate ionotropic receptors, which by causing the permeation of excess amounts of calcium ions trigger a set of deleterious cellular events leading to cell death. Indeed, while exposure of neuronal cultures to anaerobic conditions leads to neuronal destruction via excess Glutamate, almost complete protection is afforded with Glutamate receptor antagonists.

Cerebral ischemia is associated with large increases of extracellular Glutamate due in part to a calcium-dependent release from nerve terminals and the reversed transport action of Glutamate transporters. Glutamate levels rise abruptly and peak during ischemia, slowly declining to pre-ischemic levels within 10-20 min of reperfusion. It is well known that both Glutamate release inhibitors and antagonists of ionotropic Glutamate receptors protect against the pathological consequences of ischemia in both adult animals and neonates. Antagonists of ionotropic Glutamate receptors significantly reduce the size of the damaged brain area and attenuate neurological deficits. Moreover, clinical evidence has demonstrated a correlation between that the presence of increased concentrations (>8 μM) of Glutamate in the cerebrospinal fluid and the progression of the neurological deficits in stroke patients; and between prolonged release of Glutamate [up to 50 times normal levels (>20 μM)] and poor clinical outcome in severe human head trauma. Several approaches are presently under investigation for combating Glutamate-mediated excitotoxicity. These include: inhibiting Glutamate synthesis, blocking its release from presynaptic terminals, antagonizing its actions on postsynaptic receptors, and accelerating its reuptake from the synaptic cleft.

However, because of the crucial importance of Glutamatergic neurotransmission in the CNS, it is clear that, upon systemic drug administration, both Glutamate release inhibitors and the Glutamate receptor antagonists are hampered by severe undesired collateral actions at unaffected sites (healthy CNS tissue). This reduces significantly the efficacy of potential drugs affecting Glutamatergic neurotransmission: indeed, to date drugs affecting the Glutamatergic system have yet to receive FDA approval. In fact, the recent discovery that some of the Glutamatergic neuroprotective drugs highly effective in rodent models of stroke are ineffective or even deleterious in humans has lead many pharmaceutical companies to reconsider the strategy of Glutamate receptor antagonists in the treatment of neurodegenerative disorders.

Neuroprotective Immunity: In the context of neuroimmune interaction, and Glutamate effects in the CNS, the recent discovery of neuroprotective interactions between T-cells and neuronal tissue in neurotoxicity, disease and injury is intriguing. Several studies by Schwartz, et al have shown that T-cell deficient mice are more susceptible to experimentally induced neuronal injury and Glutamate neurotoxicity, and that reconstitution with wild-type splenocytes can effectively restore resistance. Additional evidence for such protective autoimmunity in CNS trauma was provided by the demonstration of potentiation of neuronal survival by prior, unrelated CNS insult in autoimmune encephalomyelitis-resistant strains of mice (see, for example, Yoles, et al, J Neurosci 2001, Jun. 1; 21(11): 3740-48; Kipnis, et al, J Neurosci 2001 Jul. 1; 21(13):4564-71; and Schori, et al, J Neuroimmunol 2001 Oct. 1:119(2):199-204). Clinical application of such neuroprotective immunity has been proposed, employing vaccination with non-pathogenic CNS derived peptides such as MBP to boost innate beneficial autoimmunity (Schwartz and Kipnis, Trends Mol Med 2001 June; 7(6):252-58; and Schwartz, Surv Ophthalmol 2001 May; 45 Suppl 3:S256-60) and stimulation of peripheral monocytes for enhancement of axonal regeneration (U.S. Pat. No. 6,117,242 to Eisenbach-Schwartz). No mention is made of Glutamate or Glutamate analog stimulation of T-cell activity, and furthermore, the authors note the substantial risk of inducing undesired autoimmune disease using immunization with self antigens.

Studies of lymphocyte activation in other neurogenic conditions also indicate a potential neuroprotective role of immune cells: in patients with encephalitis and MS, the beneficial brain-derived-neurotrophic-factor BNDF is secreted by immune cells in response to CNS auto-antigen stimulation (Kerschensteiner, et al, J Exp Med 1999 Mar. 1; 189(5):865-70). Furthermore, in clinical trials of an altered peptide ligand of myelin basic protein administered to patients with relapsing-remitting MS, reduction in lesion volume and number was achieved in the MBP-treated patients compared to the placebo group. However, the dosage required was high (5 mg), and the trial was suspended due to undesirable side effects (hypersensitivity). No mention was made of Glutamate stimulation of T-cells.

Neuroimmunology and Psychopathology: Many studies have demonstrated significant correlation between immune function and a variety of emotional and psychopathological conditions, especially schizophrenia and suicide (see, for example, Sperner-Unterweger B, et al, Scizophr Res 1999; 38:61-70; Staurenghi A H, et al Psychoneuroendocrinology 1997; 22:575-90; van Gent T, et al J Child Psychol Psychiatry 1997; 38:337-49; Nassberger L and Traskman-Bendz L Acta Psychiatr Scand 1993; 88:48-52; and Dabkowska M and Rybakowski J Psychiatr Pol 1994; 28:23-32). Presently it remains unclear whether the dysfunctional immune responses observed contribute to the psychopathogenic processes, are secondary to them, or a combination of the two.

T-cell enhancement has been observed in schizophrenia, and has been suggested as a marker of therapeutic outcome or neuroleptic treatment (Muller, et al Acta Psychiatr Scand 1993; 87:66-71 and Sperner-Unterweger B et al Scizophr Res 1999; 38:61-70). The authors made no mention of T-cell-related therapy or Glutamate modulation of T-cell activity for treatment or prevention of the abovementioned disorders.

Manipulation of immune cells for therapy of brain related disorders has been proposed by Wank (Intern Pats. WO9950393A2 and WO9950393A3 to Wank, R). Wank describes the in-vitro activation of peripheral blood monocytes (PBMC), or phagocytes, for the treatment of a variety of brain-related disorders, including psychoses, schizophrenia, autism, Down's syndrome, disturbances of cerebral development and brain injury, based on the observation of inadequate immune responses in these conditions. In a report documenting adoptive immunotherapy of patients suffering from bipolar disorder, schizophrenia or autism, Wank describes the in-vitro activation, and reintroduction of the patients' own T-cells, in order to combat “chronically infected”, understimulated lymphocytes thought associated with these disorders. In this form of therapy, the T-cells are not stimulated directly, rather via monoclonal antibodies against the CD3 polypeptide complex, and IL-2. The patients were required to endure numerous weekly treatments (up to 104 weeks in one patient), and although improvement in some symptoms was noted, additional therapies were continued during and after these trials of adoptive immunotherapy. No mention is made of direct stimulation of T-cells with neurotransmitters, of specific T-cell response to therapy, or of treatment with Glutamate or Glutamate analogs.

There is thus a widely recognized need for, and it would be highly advantageous to have methods and compositions for direct modulation of T-cell activity by the action of Glutamate and Glutamate functional analogs.

More particularly, there is a long felt need for methods and compositions for the treatment of viral and other infectious diseases, prevention and treatment of neurological disease, psychopathology, neuronal damage in CNS disease, infection and injury, containment of auto-immune and other injurious inflammatory processes, enhancement of anti-tumor immune surveillance and prevention of host rejection of engrafted tissue employing Glutamate receptor-mediated regulation of T-cell gene expression, cytokine secretion, adhesion and migration devoid of the above limitations.

### SUMMARY OF THE INVENTION

According to the present invention there is provided an assay of determining the sensitivity of a T-cell to Glutamate or a Glutamate analog, the assay comprising exposing the T-cell to one or more concentrations of Glutamate or a Glutamate analog; and assessing a stimulatory state of said cell.

According to further features in the described preferred embodiments exposing said T-cell is performed in vivo and/or in vivo.

According to yet further features in the described preferred embodiments said T-cell is a resting or stimulated T cell.

According to yet another aspect of the present invention, there is provided an assay of determining an effect of Glutamate or a Glutamate analog on a T-cell related disease or condition, the assay comprising exposing an organism having the T-cell related disease or condition to at least one concentration of Glutamate or a Glutamate analog; and assessing at least one T-cell related symptom in said organism.

According to further features in the described preferred embodiments said Glutamate analog is selected from the group consisting of naturally occurring and synthetic analogs.

According to still further features in the described preferred embodiments said Glutamate analog is an upregulator or a downregulator of T cell activation.

According to still another aspect of the present invention there is provided an assay of determining the sensitivity of a T-cell to an anti-Glutamate receptor antibody, the assay comprising exposing the T-cell to an anti-Glutamate receptor antibody; and assessing a stimulatory state of said cell.

According to further features in the described preferred embodiments exposing said T-cell is performed in vivo and/or in vivo.

According to yet further features in the described preferred embodiments said T-cell is a resting or stimulated T cell.

According to an additional aspect of the present invention there is provided an assay of determining an effect of an anti-Glutamate receptor antibody on a T-cell related disease or condition, the assay comprising exposing an organism having the T-cell related disease or condition to the anti-Glutamate receptor antibody; and assessing at least one T-cell related symptom in said organism.

According to further features in the described preferred embodiments said anti-Glutamate receptor antibody is a monoclonal or a polyclonal antibody.

According to further features in the described preferred embodiments said anti-Glutamate receptor antibody is an upregulator or a downregulator of T cell activation.

According to a further aspect of the present invention there is provided an assay of determining a sensitivity of a T-cell to an expressible polynucleotide encoding a Glutamate receptor, the assay comprising introducing into the T-cell an expressible polynucleotide encoding a Glutamate receptor; and assessing a stimulatory state of said cells.

According to further features in the described preferred embodiments said T-cell is a resting or stimulated T cell.

According to yet a further aspect of the present invention there is provided an assay of determining an effect of an expressible polynucleotide encoding a Glutamate receptor on a T-cell related disease or condition, the assay comprising introducing into one or more tissues of an organism having the T-cell related disease or condition to an expressible polynucleotide encoding a Glutamate receptor, and assessing at least one T-cell related symptom in said organism.

According to further features in the described preferred embodiments said expressible polynucleotide being capable of transient or stable expression.

According to a further aspect of the present invention there is provided an assay of determining the sensitivity of a T-cell to a polynucleotide that downregulates Glutamate receptor expression, the assay comprising introducing into the stimulated T cell the polynucleotide that down-regulates Glutamate receptor expression; and assessing a stimulatory state of said T-cell.

According to a yet another aspect of the present invention there is provided an assay of determining an effect of a polynucleotide that downregulates Glutamate receptor expression on a T cell related disease or condition, the assay comprising introducing into at least one T cell related tissue of an organism having the T cell related disease or condition the polynucleotide that downregulates Glutamate receptor expression; and assessing at least one T cell related symptom in said organism.

According to further features in the described preferred embodiments said polynucleotide is a ribozyme having specific Glutamate receptor transcript cleaving capability.

According to still further features in the described preferred embodiments said polynucleotide is an expressible polynucleotide encoding a ribozyme having specific Glutamate receptor transcript cleaving capability.

According to yet further features in the described preferred embodiments said polynucleotide comprises nucleotide sequences complementary to, and capable of binding to Glutamate receptor transcripts, coding sequences and/or promoter elements.

According to further features in the described preferred embodiments said polynucleotide is an expressible polynucleotide encoding nucleotide sequences complementary to, and capable of binding to Glutamate receptor transcripts, coding sequences and/or promoter elements.

According to further features in the described preferred embodiments introducing said polynucleotide and/or expressible polynucleotide is performed in vivo or ex vivo.

According to a yet another aspect of the present invention there is provided a method of modulating T cell activity, the method comprising exposing T-cells to Glutamate or a T cell activity modulating Glutamate analog.

According to further features in described preferred embodiments exposing said T cells to said Glutamate or said T-cell activity modulating Glutamate analog is performed in vitro or in vivo.

According to still further features in described preferred embodiments said T-cell activity modulating Glutamate analog is an upregulator, causing increased T-cell activity, or a downregulator, causing decreased T-cell activity.

According to yet further features in described preferred embodiments said Glutamate analog is selected from the group consisting of naturally occurring and synthetic analogs.

According to further features in described preferred embodiments said downregulator is a Glutamate receptor blocker.

According to a yet another aspect of the present invention there is provided a method of modulating T cell activity, the method comprising exposing T-cells to a T cell activity modulating antibody, wherein said antibody modulates T cell responsiveness to Glutamate or a Glutamate analog.

According to further features in described preferred embodiments exposing said T cells to said T-cell activity modulating antibody is performed in vitro or in vivo.

According to still further features in described preferred embodiments said T-cell activity modulating antibody is an upregulator, causing increased T-cell activity, or a downregulator, causing decreased T-cell activity.

According to yet further features in described preferred embodiments said T-cell activity modulating antibody is an anti-Glutamate receptor antibody.

According to still further features in described preferred embodiments said T-cell activity modulating antibody is a monoclonal or polyclonal antibody.

According to still another aspect of the present invention there is provided a method of upregulating T-cell activity in a mammalian subject, the method comprising administering to the subject a therapeutically effective amount of Glutamate or a T-cell upregulating Glutamate analog, said amount being sufficient to upregulate T cell activity in the mammalian subject.

According to further features in described preferred embodiments said upregulating Glutamate analog is selected from the group consisting of naturally occurring and synthetic analogs.

According to yet further features in described preferred embodiments administering said therapeutically effective amount of Glutamate or a T cell upregulating Glutamate analog is performed in vivo or ex vivo.

According to still another aspect of the present invention there is provided a method of upregulating T-cell activity in a mammalian subject, the method comprising administering to the subject a therapeutically effective amount of an upregulating anti-Glutamate receptor antibody, said amount being sufficient to stimulate Glutamate receptor activation, thereby upregulating T cell activity in the mammalian subject.

According to further features in described preferred embodiments administering said therapeutically effective amount of an upregulating anti-Glutamate receptor antibody is performed in vivo or ex vivo.

According to still further features in described preferred embodiments said upregulating anti-Glutamate receptor antibody is a monoclonal or polyclonal antibody.

According to a yet another aspect of the present invention there is provided a method of upregulating T-cell activity in a mammalian subject, the method comprising introducing into at least one T cell related tissue of the subject an expressible polynucleotide encoding a Glutamate receptor, said expressible polynucleotide being capable of enhancing Glutamate receptor expression in said T cells, thereby upregulating T-cell activity in the mammalian subject.

According to further features in described preferred embodiments introducing said expressible polynucleotide is performed in vivo or ex vivo.

According to still further features in described preferred embodiments said expressible polynucleotide contains a sequence as set forth in any of SEQ ID NOs:1 and 2.

According to yet further features in described preferred embodiments said expressible polynucleotide contains a sequence at least 60% homologous to SEQ ID NOs:1 and 2.

According to still further features in described preferred embodiments said expressible polynucleotide being capable of transient or stable expression.

According to further features in described preferred embodiments said subject is suffering from a T cell related disease or condition selected from the group consisting of congenital immune deficiencies, acquired immune deficiencies, infection, neurological disease and injury, psychopathology and neoplastic disease.

According to still another aspect of the present invention there is provided a method of downregulating T-cell activity in a mammalian subject, the method comprising administering to the subject a therapeutically effective amount of a T-cell downregulating Glutamate analog, said amount being sufficient to downregulate T cell activity, thereby downregulating said T cell activity in the mammalian subject.

According to further features in described preferred embodiments said downregulator is a Glutamate receptor blocker.

According to yet further features in described preferred embodiments said Glutamate analog is selected from the group consisting of naturally occurring and synthetic analogs.

According to still another aspect of the present invention there is provided a method of downregulating T-cell activity in a mammalian subject, the method comprising administering to the subject a therapeutically effective amount of a downregulating anti-Glutamate receptor antibody, said amount being sufficient to block Glutamate receptor activation, thereby down-regulating T cell activity in the mammalian subject.

According to still further features in described preferred embodiments said downregulating anti-Glutamate receptor antibody is a monoclonal or polyclonal antibody.

According to still further features in described preferred embodiments administering said therapeutically effective amount of a T cell down-regulating Glutamate analog or downregulating anti-Glutamate receptor antibody is performed in vivo or ex vivo.

According to a yet another aspect of the present invention there is provided a method of downregulating T-cell activity in a mammalian subject, the method comprising introducing into at least one T cell related tissue of the subject a polynucleotide which downregulates Glutamate receptor expression, said polynucleotide being capable of reducing sensitivity to Glutamate activation, thereby downregulating T-cell activity in the mammalian subject.

According to further features in described preferred embodiments said polynucleotide is a ribozyme having specific Glutamate receptor transcript cleaving capability.

According to yet further features in described preferred embodiments polynucleotide is an expressible polynucleotide encoding a ribozyme having specific Glutamate receptor transcript cleaving capability.

According to still further features in described preferred embodiments said polynucleotide comprises nucleotide sequences complementary to, and capable of binding to Glutamate receptor transcripts, coding sequences and/or promoter elements.

According to further features in described preferred embodiments said polynucleotide is an expressible polynucleotide encoding nucleotide sequences complementary to, and capable of binding to Glutamate receptor transcripts, coding sequences and/or promoter elements.

According to further features in described preferred embodiments introducing said polynucleotide is performed in vivo or ex vivo.

According to still further features in described preferred embodiments said subject is suffering from a T cell related disease or condition selected from the group consisting of autoimmune, allergic, neoplastic, hyperreactive, psychopathological and neurological diseases and conditions; graft-versus-host disease, and allograft rejection.

According to still another aspect of the present invention there is provided a method of preventing or treating a cancerous disease or condition in a mammalian subject, the method comprising administering to the subject a therapeutically effective amount of a downregulating Glutamate analog, said amount being sufficient for effectively blocking Glutamate activity, thereby causing a reduction in cancer cell proliferation and/or metastasis in the mammalian subject.

According to further features in described preferred embodiments said downregulating Glutamate analog is a naturally occurring or synthetic analog.

According to still another aspect of the present invention there is provided a method of preventing or treating a cancerous disease or condition in a mammalian subject, the method comprising administering to the subject a therapeutically effective amount of a downregulating anti-Glutamate receptor antibody, said amount being sufficient for effectively blocking Glutamate activity, thereby causing a reduction in cancer cell proliferation and/or metastasis in the mammalian subject.

According to further features in described preferred embodiments administering said downregulating Glutamate analog or anti-Glutamate antibody is performed in vivo or ex vivo.

According to yet further features in described preferred embodiments said anti-Glutamate receptor antibody is a monoclonal or polyclonal antibody.

According to still another aspect of the present invention there is provided a method of preventing or treating a cancerous disease or condition in a mammalian subject, the method comprising introducing into at least one T cell related tissue of the subject a polynucleotide which specifically inhibits Glutamate receptor production, said polynucleotide being capable of effectively reducing sensitivity to Glutamate stimulation, thereby causing a reduction in cancer cell proliferation and/or metastasis in the mammalian subject.

According to further features in described preferred embodiments said polynucleotide is a ribozyme having specific Glutamate receptor transcript cleaving capability.

According to yet further features in described preferred embodiments said polynucleotide is an expressible polynucleotide encoding a ribozyme having specific Glutamate receptor transcript cleaving capability.

According to still further features in described preferred embodiments said polynucleotide comprises nucleotide sequences complementary to, and capable of binding to Glutamate receptor transcripts, coding sequences and/or promoter elements.

According to further features in described preferred embodiments said polynucleotide is an expressible polynucleotide encoding nucleotide sequences complementary to, and capable of binding to Glutamate receptor transcripts, coding sequences and/or promoter elements.

According to still further features in described preferred embodiments introducing said polynucleotide is performed in vivo or ex vivo.

According to yet further features in described preferred embodiments said cancerous disease or condition is a myeloproliferative disease.

According to another aspect of the present invention there is provided a method of relieving or preventing a T-cell related disease or condition in a mammalian subject, the method comprising administering to said subject a therapeutically effective amount of downregulating anti-Glutamate receptor antibody, said amount being sufficient to reduce Glutamate stimulation of T cell activity, thereby alleviating said T cell related disease or condition in the mammalian subject.

According to further features in described preferred embodiments said anti-Glutamate receptor antibody is a monoclonal or polyclonal antibody.

According to still further features in described preferred embodiments administering said therapeutically effective amount of said down-regulating anti-Glutamate receptor antibody is performed in vivo or ex vivo.

According to a still another aspect of the present invention there is provided a method of relieving or preventing a T cell related in a mammalian subject, the method comprising introducing into at least one T cell related tissue of the subject a polynucleotide which specifically inhibits Glutamate receptor production, said polynucleotide being capable of effectively reducing sensitivity to Glutamate stimulation, thereby alleviating said T cell related condition or disease in the mammalian subject.

According to further features in preferred embodiments introducing said polynucleotide is performed in vivo or ex vivo.

According to yet further features in preferred embodiments said polynucleotide is a ribozyme having specific Glutamate receptor transcript cleaving capability.

According to further features in preferred embodiments said polynucleotide is an expressible polynucleotide encoding a ribozyme having specific Glutamate receptor transcript cleaving capability.

According to yet further features in preferred embodiments said polynucleotide comprises nucleotide sequences complementary to, and capable of binding to Glutamate receptor transcripts, coding sequences and/or promoter elements.

According to still further features in preferred embodiments said polynucleotide is an expressible polynucleotide encoding nucleotide sequences complementary to, and capable of binding to Glutamate receptor transcripts, coding sequences and/or promoter elements.

According to a yet another aspect of the present invention there is provided a method of relieving or preventing a T cell related disease or condition in a mammalian subject, the method comprising administering to the subject a therapeutically effective amount of downregulating Glutamate analog, said amount being sufficient to reduce T cell activity, thereby alleviating said T cell related disease or condition in the mammalian subject.

According to further features in preferred embodiments said downregulating Glutamate analog is a Glutamate receptor blocker.

According to still further features in described preferred embodiments said mammalian subject is suffering from a T-cell related pathology selected from the group consisting of autoimmune, allergic, neoplastic, hyperreactive, psychopathological and neurological diseases and conditions; graft-versus-host disease, and allograft rejection.

According to still another aspect of the present invention there is provided a method of relieving or preventing a T cell related disease or condition in a mammalian subject, the method comprising administering to the subject a therapeutically effective amount of Glutamate or an upregulating Glutamate analog, said amount being sufficient to stimulate T cell activity, thereby alleviating said T cell related disease or condition in the mammalian subject.

According to further features in preferred embodiments administering said therapeutically effective amount of Glutamate or downregulating or upregulating Glutamate analog is performed in vivo or ex vivo.

According to yet further features in preferred embodiments said downregulating or upregulating Glutamate analog is a naturally occurring or synthetic analog.

According to yet another aspect of the present invention there is provided a method of relieving or preventing a T cell related condition or disease in a mammalian subject, the method comprising introducing into at least one T cell related tissue of the subject an expressible polynucleotide encoding a Glutamate receptor, said polynucleotide being capable of enhancing Glutamate receptor expression in said T cells, thereby alleviating said T cell related disease or condition in the mammalian subject.

According to further features in preferred embodiments introducing said expressible polynucleotide is performed in vivo or ex vivo.

According to still further features in described preferred embodiments said expressible polynucleotide contains a sequence as set forth in any of SEQ ID NOs:1 and 2.

According to yet further features in described preferred embodiments said expressible polynucleotide contains a sequence at least 60% homologous to SEQ ID NOs:1 and 2.

According to still further features in described preferred embodiments said expressible polynucleotide being capable of transient or stable expression.

According to further features in described preferred embodiments said subject is suffering from a T cell related disease or condition selected from the group consisting of congenital immune deficiencies, acquired immune deficiencies, infection, neurological disease and injury, psychopathology and neoplastic disease.

According to still another aspect of the present invention there is provided a pharmaceutical composition comprising as an active ingredient Glutamate or at least one upregulating Glutamate analog, being packaged and indicated for use in the prevention and/or treatment of a T cell related condition, in which stimulating T-cell activity is an effective therapy.

According to yet another aspect of the present invention there is provided a pharmaceutical composition comprising as an active ingredient at least one downregulating Glutamate analog, being packaged and indicated for use in the prevention and/or treatment of a T cell related condition, in which inhibiting T-cell activity is an effective therapy.

According to another aspect of the present invention there is provided a pharmaceutical composition comprising as an active ingredient an upregulating anti-Glutamate receptor antibody, being packaged and indicated for use in the prevention and/or treatment of a T cell related condition, in which stimulating T cell activity is an effective therapy.

According to still another aspect of the present invention there is provided a pharmaceutical composition comprising as an active ingredient a downregulating anti-Glutamate receptor antibody, being packaged and indicated for use in the treatment of a T cell related condition, in which inhibiting T-cell activity is an effective therapy.

The present invention successfully addresses the shortcomings of the presently known configurations by providing methods for direct modulation of T-cell activity by the action of neurotransmitter and specific neurotransmitter receptor functional analogs and, more particularly methods for the treatment of viral and other infectious diseases, containment of auto-immune and other injurious inflammatory processes, inhibition and prevention of tumor growth and dissemination, and prevention of host rejection of engrafted tissue. Specifically, the present invention employs Glutamate receptor-mediated regulation of expression of T cell genes, activation, adhesion, migration and, ultimately, T-cell participation in inflammation and surveillance in infection and disease. One important aspect of the present invention is the ability of the neurotransmitter and functional analogs thereof to act in immune-privileged environments, such as the brain. Similarly, inhibition of Glutamate receptor-mediated T cell activation is proposed for the limitation and prevention of metastatic spread of T-cell-related and other cancerous conditions.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-1D are tables illustrating the Glutamate-induced regulation of specific gene expression in the human T-cell, through analysis of gene expression using the atlas human cDNA expression array. For the atlas array analysis, ³²P-labeled cDNA was prepared from poly A+ RNA, isolated from normal human T cells that had been treated with or without 10 nM of Glutamate for 72 hours. The cDNA was hybridized to the atlas membranes according to the manual, and expression was visualized by autoradiography. FIG. 1A is a table listing genes upregulated by Glutamate. FIGS. 1B-D is a table of genes downregulated by Glutamate. Note the enhanced expression of T-cell genes such as Rapamycin-selective-25 KD Immunophilin (FKBP-25), Heat shock protein 40 (Hsp 40), Cathepsin E presursor, Carbonic anhydrase-related protein (carp) (ca-viii), peptidyl-glycine alpha-amidating monooxygenase presursor (pam), and axonemal dynein heavy chain (fragment) as well as of genes whose expression has been previously undetected in T cells such as Stimulator of Fe Transport (SFT), oviductal glycoprotein, Clathrin light chain, Protein Inhibitor of Activated STAT (PIAS) and Cartilage Intermediate Layer Protein (CILP) (FIG. 1A).

FIG. 1E illustrates the specific induction of the human protease inhibitor BOMAPIN expression in human peripheral T-cells treated with Glutamate (10 nM) for 72 hours, employing quantitative RT-PCR assay of RNA from Glutamate-treated peripheral human T-cells. PCR was performed for 30 cycles. The ethidium bromide bands corresponding to the amplified bomapin transcripts were quantified by AlphaEase program (Alpha Innotech, San Leandro, Calif., USA). Each PCR tube contained four oligonucleotides primers, two for the Bomapin and two for the internal control (S-14). The Bomapin mRNA level in the Glutamate treated cells is about 6 fold higher than that of the untreated (Untreated) cells. Note the inhibition of Glutamate-mediated Bomapin expression by the specific ionotropic glutamate receptor antagonist: CNQX (Glutam./CNQX).

FIG. 2 illustrates the induction by Glutamate of “typical” cytokine secretion in resting cloned human T-cells. Cloned resting human Th2 cells (clone 401) were incubated for 20 hours with 10 nM Glutamate (Glutamate) or no addition (untreated), and levels of the cytokine IL-4 were measured in the supernatants by a qualitative sandwich ELISA, as described in Materials and Methods. The results are expressed as pg/ml IL-4. Note the clear induction of IL-4 secretion in the Glutamate-treated cells, absent in the non-activated cells.

FIG. 3 illustrates the induction by Glutamate of “forbidden” cytokine secretion in resting cloned human T-cells. Cloned resting human Th1 cells (clone 305) were incubated for 20 hours with 10 nM Glutamate (Glutamate) or no addition (untreated), and levels of the Th2-specific cytokine IL-10 were measured in the supernatants by a qualitative sandwich ELISA, as described in Materials and Methods. The results are expressed as pg/ml IL-10.

FIG. 4 illustrates the induction by Glutamate of “forbidden” cytokine secretion in resting cloned human T-cells. Cloned resting human Th1 cells (clone 305) were incubated for 20 hours with 10 nM Glutamate (Glutamate) or no addition (untreated), and levels of the Th2-specific cytokine IL-4 were measured in the supernatants by a qualitative sandwich ELISA, as described in Materials and Methods. The results are expressed as pg/ml IL-4.

FIG. 5 illustrates the induction by Glutamate of Interferon-γ secretion in activated cloned human T-cells. Cloned human Th0 cells (clone 234) were activated (alloprimed) by incubation for 20 hours with a fully mismatched (in its MHC-class I and II) EBV-transformed B cell line (allogeneic specific feeder layer) alone (Activated) or in the presence of additional 10 nM Glutamate (Activated+Glutamate), and levels of the cytokine IFN-γ were measured in the supernatants by a qualitative sandwich ELISA, as described in Materials and Methods. The results are expressed as pg/ml IFN-γ.

FIG. 6 illustrates the induction by Glutamate of Interferon-γ secretion in activated cloned human T-cells. Cloned resting human Th1 cells (clone 305) were activated (alloprimed) by incubation for 20 hours with fully mismatched (in its MHC-class I and II) EBV-transformed B cell line (allogeneic specific feeder layer) alone or with additional 10 nM Glutamate, and levels of the cytokine IFN-γ were measured in the supernatants by a qualitative sandwich ELISA, as described in Materials and Methods. The results are expressed as pg/ml IFN-γ.

FIGS. 7A and 7B illustrate Glutamate induction of human T-cell adhesion to extracellular matrix proteins fibronectin and laminin. Normal T-cells purified from human blood samples were pretreated (30 min. at 37° C.) with either Glutamate alone (10⁻⁸ M) or Glutamate and the AMPA-specific antagonist CNQX (10⁻⁸ Glu+CNQX) and then tested for their adhesion to fibronectin (FN) in FN-coated microtiter wells (FIG. 7A) or laminin, in laminin coated microtiter wells (FIG. 7B). Adhesion of untreated cells (BG) serves as a control. The results are expressed as the OD₄₀₅ of the lysed, fibronectin- or laminin-adherent cells remaining after incubation and repeated washings. Note that even a low concentration of Glutamate (10⁻⁸ M) induced significant T-cell adhesion to fibronectin and laminin, while the Glutamate receptor (AMPA) antagonist CNQX reversed Glutamate's effect.

FIG. 8 illustrates the Glutamate-induced migration of normal human T-cells towards the chemokine SDF-1. Human T-cells purified from fresh blood samples of different human donors were pretreated (18 h-24 hours at 37° C.) with Glutamate (10⁻⁸M), labeled with a fluorescent dye, and tested for their migration towards the chemokine-SDF-1 in a chemotaxis microchamber. The cells in each experimental group were counted by FACSORT. The results are expressed as the number of fluorescently-labeled migrating Glutamate-treated T-cells vs. untreated controls (Untreated). Note that even exceedingly low concentrations (10⁻⁸M) of Glutamate can directly induce the migration of normal human T-cells towards the SDF-1 chemokine.

FIG. 9 illustrates the inhibition of human T-cell Glutamate receptor expression by Glutamate. Normal human T-cells purified from fresh blood samples were incubated with Glutamate (10⁻⁵ M), and surface GluR3 receptor measured in samples by double immunofluorescence assay at indicated intervals (5-60 minutes). Inhibition of GluR3 expression is expressed as the percentage of GluR3-positive cells from total T-cells, divided by the baseline (Untreated cells, 0 minutes) percentage. Note the rapid, time-dependent inhibition of GluR3 expression with Glutamate, reaching 80% of baseline at 20 minutes.

FIGS. 10A and 10B depict the inhibition of human T-cell GluR3 receptor gene expression by Glutamate. FIGS. 10A and 10B demonstrate the absence of RT-PCR-amplified GluR3 Glutamate receptor cDNA transcripts in normal human T-cells treated with Glutamate for 24 hours (Glutamate 10 mM) after 1.5% agarose gel electrophoresis and ethidium bromide staining, indicating that Glutamate inhibits human T-cell GluR3 receptor expression at the level of gene transcription. FIGS. 10A and 10B are ethidium bromide stained gels of RT-PCR amplified cDNA from two similar experiments. Note the absence of the slower-migrating band of GluR3 cDNA from the Glutamate treated cells (Glutamate 10 mM).

### DESCRIPTION OF THE PREFERRED EMBODIMENTS

The present invention is of novel methods for the direct modulation of T-cell activity by the action of Glutamate and Glutamate functional analogs and, more particularly, to methods for the treatment of infectious diseases, inhibition and prevention of tumor growth and dissemination, prevention and treatment of neurological disease, psychopathology, neuronal damage in CNS disease, infection and injury, containment of auto-immune and other injurious inflammatory processes, enhancement of anti-tumor immune surveillance and prevention of host rejection of engrafted tissue. Specifically, the present invention employs GluR3 Glutamate receptor-mediated regulation of gene expression and cytokine secretion, in turn effecting integrin-mediated adhesion, chemotactic migration and, ultimately, T-cell participation in inflammation and surveillance in infection, injury and disease.

The principles and operation of methods and compositions for the modulation of T-cell activity by the action of Glutamate and Glutamate functional analogs according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

At any given moment, T-cell populations throughout the body have to carry out a myriad of different activities, among them patrolling and surveillance, helping and suppressing, combating and killing. Moreover, T-cell activities must be precisely regulated and coordinated with many other cell types in general, and, perhaps most importantly, with dynamic neuro-endocrine networks. It is difficult to conceive that all these tasks are mediated solely via the ‘classical’ immunological interactions between the T-cell receptor (TCR), the principal receptor of these cells, and specific antigens, even if assisted by other immunological molecules, such as cytokines and chemokines and their receptors. In fact, the factors responsible for regulating T-cell activities within immune privileged environments, such as the brain, are still unknown and their discovery will certainly have important implications for the understanding and treatment of various T-cell mediated CNS pathologies, such as the autoimmune T-cell mediated multiple sclerosis.

Can T-cells respond directly to neurotransmitter molecules, despite the conceptual dogma of a ‘language’ barrier between effector molecules used for communication within the nervous, endocrine and immune systems? No doubt that such a direct mode of communication could be of great benefit for coordinating body functions in numerous physiological and pathophysiological conditions. While reducing the present invention to practice, this question was addressed by investigating whether T-cells can be directly activated by the amino acid Glutamate, a ubiquitous excitatory neurotransmitter of the mammalian nervous system.

Glutamate and its receptors are responsible for numerous physiological, and pathological functions within the nervous system. Thus, Glutamate and its receptors are crucial to the neurophysiology of learning and memory, for example, and also the primary source of neurotoxicity and neuronal damage in pathological states such as stroke, trauma and epileptic seizure. The results presented herein show that Glutamate indeed activates normal human T-cells, in the absence of any additional stimuli, inducing “typical” and “forbidden” cytokine secretion profiles, regulates expression of specific T-cell genes, triggers T-cell binding to fibronectin, and stimulates chemokine-mediated chemotaxis.

It will be appreciated, in the context of the present invention, that while the abovementioned effects of Glutamate are mediated by glutamate receptors expressed in the nervous system, primarily on neurons and glia cells, the presence and function of specific glutamate receptors on cells of the immune system has not been previously demonstrated. Surprisingly, while reducing the present invention to practice, the inventor uncovered, for the first time, that normal human T-cells, human T-leukemia-cells, and mouse anti-myelin-basic-protein T-cells express high levels of glutamate ion channel receptor (ionotropic) of the AMPA subtype-3 (GluR3). The evidence for GluR3 on T-cells includes GluR3-specific RT-PCR, western blot, immunocytochemical-staining and flow-cytometry. Sequencing showed that the T-cell expressed GluR3 is identical to the brain GluR3.

The results presented herein show that Glutamate (10 nM), in the absence of any additional molecule, triggered T-cell function: integrin-mediated T-cell adhesion to laminin and fibronectin, a function normally performed by activated T-cells only. The effect of glutamate was mimicked by AMPA receptor-agonists, and blocked specifically by the selective receptor-antagonists CNQX and NBQX, and by relevant anti-integrin monoclonal-antibodies. Glutamate also increased the CXCR4-mediated T-cell chemotactic-migration towards the key chemokine CXCL12/SDF-1. In addition, the results presented herein show that Glutamate indeed activates normal human T-cells, in the absence of any additional stimuli, inducing “typical” and “forbidden” cytokine secretion profiles, and can trigger the gene expression of specifc genes while supressing the expresion fo others. Finally, Glutamate was found herein to regulate the levels of expression of its own receptor, GluR3, both on the mRNA level and on the level of the receptor protein expressed on the membrane of human T-cells. This is the first demonstration of direct activation by Glutamate of T-cell function and gene expression.

While reducing the present invention to practice, an increased secretion of T-cell specific cytokines was directly induced by Glutamate in both unstimulated and stimulated human T-cells, demonstrating, for the first time, the presence of specific Glutamate receptors and Glutamate-mediated activation in T-cells. In addition, similarly low, physiological levels of Glutamate induced greatly enhanced T-cell fibronectin adhesion and chemotactic, cytokine (SDF-1alpha)-mediated migration. Thus, under normal conditions, Glutamate may lead to beneficial activation and migration of T-cells towards resting, inflamed, injured or stressed tissues, and may serve for direct neural coordination of immune function. Furthermore, under conditions of undesirable T-cell migration and function (autoimmune disease, chronic inflammation, allergic conditions, graft-versus-host disease, and allograft rejection) Glutamate may have detrimental effects and may be a target for immunosuppression.

While further reducing the present invention to practice, it was found that similarly low, physiological concentrations of Glutamate (10⁻⁸ M) induce expression of the protease inhibitor Bomapin in normal human T-cells. Since Bomapin has been associated with resistance to TNF-alpha-induced apoptosis of cells (Schleer R R and Chuang T L, J Biol Chem 2000; 275:26385-89), Glutamate stimulation of T-cells may reduce susceptibility to apoptosis, thus enhancing T-cell longevity and effectiveness.

In the context of the present invention, it is important to note the role of immune function in general, and T-cells in particular, in neuroprotective immunity. Activated T-cells in sufficient numbers, at crucial locations in the CNS, and with appropriate temporal coordination, are necessary for optimal healing following neuronal injury or viral infection of the CNS (Yoles E et al J Neurosci 2000; 21:3740-8; and Binder G K and Griffin D E Science 2001; 293:303-6). Thus, the compositions and methods of the present invention can be used for treatment and prevention of neuronal damage in CNS injury and infection.

In addition, while reducing the present invention to practice, it was unexpectedly observed that Glutamate swiftly downregulates the transcription and surface expression of the GluR3 Glutamate receptor in normal human T-cells. Thus, the results presented herein reveal a novel mechanism by which the neurotransmitter Glutamate can, by itself, modulate the synthesis and surface expression of a specific, feedback-related Glutamate receptor, and directly affect the T-cells potential for activation.

The present invention provides methods and compositions for specific neurotransmitter-mediated regulation of T-cell function via modulation of cytokine secretion, T-cell adhesion, chemokine-mediated migration, receptor expression and sensitivity to stimulation.

Diseases or conditions related to T-cell deficiency or dysfunction would require upregulation of T-cell function, by Glutamate analogs possessing agonist or stimulatory properties. Although therapeutic use of Glutamate and agonist analogs of Glutamate has been previously disclosed (see, for example U.S. Pat. No. 6,211,245 to Meuller, et al; U.S. Pat. Nos. 6,109,269 and 6,227,203 to Rise, et al, and U.S. Pat. No. 6,094,598 to Elsberry, et al), the disclosed applications have all targeted neuronal excitatory Glutamate receptors for stimulation in deficiency disorders such as addiction, movement deficiency, and reward deficiency syndrome. No mention has been made of Glutamate modulation of neuroimmune functions, or of Glutamate-mediated activation of T-cell function.

Thus, according to the present invention there is provided a method of modulating T-cell activity, the method effected by exposing T-cells to Glutamate or a T-cell activity modulating Glutamate analog. In one embodiment of the invention, the Glutamate analog is an upregulator, causing increased T-cell activity. The Glutamate upregulating analog may be a naturally occurring or synthetic analog. In one preferred embodiment of the present invention, the upregulating Glutamate analog is an ionotrophic upregulator. In another preferred embodiment, the analog is a GluR3-specific agonist alpha-amino-3-hydroxy-5-methyl-4-isoxazolepropionic acid (AMPA). Commercially available upregulating Glutamate analogs suitable for use in the compositions and methods of the present invention may include, but are not limited to (RS) AMPA, (S) AMPA, L-ODAP, L-Quisqualic acid, IDRA-21 and NAAG Peptidase inhibitor (see, for example, Neurochemicals, Calbiochem Catalog, Calbiochem, Calif.).

T-cells may be isolated from the blood by procedures known to one skilled in the art (see, for example, the Materials and Methods section that follows). Thus, in the method of the present invention T-cells may be exposed to the Glutamate, or the upregulating analog in vivo, by administration to the subject via intravenous, parenteral, oral, transdermal, intramuscular, intranasal or other means or in vitro, after removal of T-cells from the body and their isolation.

A specific example of ex vivo treatment of immune cells for activation and therapeutic readministration may be found in Intn'l Pat. No. WO9950393A2 and A3 to Wank, although the methods described differ significantly from the methods disclosed herein. Wank describes the isolation and in vitro activation of peripheral blood mononuclear cells (phagocytes) from patients suffering from brain-related diseases, disorders and damage, including psychoses, autism, schizophrenia and developmental disturbances. In a report documenting adoptive immunotherapy of patients suffering from bipolar disorder, schizophrenia or autism, Wank describes similar in-vitro activation, and reintroduction of the patients' own T-cells, in order to combat “chronically infected”, understimulated lymphocytes thought associated with these disorders. In this form of therapy, the T-cells are not stimulated directly, rather via monoclonal antibodies against the CD3 polypeptide complex, and IL-2. The patients were required to endure numerous weekly treatments (up to 104 weeks in one patient), and although improvement in some symptoms was noted, additional therapies were continued during and after these trials of adoptive immunotherapy. No mention is made of direct stimulation of T-cells with neurotransmitters, of specific T-cell response to therapy, or of treatment with Glutamate or Glutamate analogs.

Further according to the present invention there is provided a method of upregulating T-cell activity in a mammalian subject, the method effected by administering to the subject a therapeutically effective amount of Glutamate or a T-cell upregulating Glutamate analog thereby upregulating T-cell activity in the mammalian subject. In the method of the present invention the Glutamate, or the upregulating analog may be administered in vivo, by administration to the subject via intravenous, parenteral, oral, transdermal, intramuscular, intranasal or other means or in vitro, after removal of T-cells from the body and their isolation.

Cell surface receptors may be targeted by specific antibodies, binding to epitopes exposed to the cellular environment. Although these antibodies may block ligand-receptor interaction, in binding some may also activate signal transduction pathways, behaving as agonists: this is commonly seen in autoimmune disease, such as Graves disease and pemphigus (for example, see Grando, S A. Antireceptor activity in pemphigus. Dermatology 2000; 201(4) 290-295; and Mijares, A., Lebesque, D., Walluk G. and Hoebeke, J. From agonist to antagonist. Mol. Pharmacol. 2000 August 58 (2): 373-378). Similarly, specific antibodies directed against T-cell Glutamate receptors may act as agonists, stimulating T-cell activity.

Thus, according to the present invention there is provided a method of modulating T-cell activity, the method effected by exposing the T-cells to an upregulating anti-Glutamate receptor antibody. T-cells may be exposed to the antibody in vivo or isolated from the organism and exposed in vitro (for methods of T-cell activation in vitro see, for example, T-cell receptor activation, binding or in-vitro migration assay in Materials and Methods section below).

As is used herein, the term “antibody” refers to either a polyclonal or monoclonal antibody, recognizing at least one epitope of Glutamate receptor. The present invention can utilize serum immunoglobulins, polyclonal antibodies or fragments thereof, (i.e., immunoreactive derivative of an antibody), or monoclonal antibodies or fragments thereof.

The term “antibody” as used in this invention includes intact molecules as well as functional fragments thereof, such as Fab, F(ab′)2, and Fv that are capable of binding to macrophages. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of making these fragments are known in the art. (See for example, Harlow and Lane, Antibodies: A Laboratory Manual, Cold Spring Harbor Laboratory, New York, 1988, incorporated herein by reference).

As used in this invention, the term “epitope” means any antigenic determinant on an antigen to which the paratope of an antibody binds.

Epitopic determinants usually consist of chemically active surface groupings of molecules such as amino acids or carbohydrate side chains and usually have specific three dimensional structural characteristics, as well as specific charge characteristics.

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R., Biochem. J., 73: 119-126, 1959. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al., Proc. Nat'l Acad. Sci. USA 69:2659-62, 1972. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow and Filpula, Methods, 2: 97-105, 1991; Bird et al., Science 242:423-426, 1988; Pack et al., Bio/Technology 11:1271-77, 1993; and Ladner et al., U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry, Methods, 2: 106-10, 1991.

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368 812-13 (1994); Fishwild et al., Nature Biotechnology 14, 845-51 (1996); Neuberger, Nature Biotechnology 14, 826 (1996); Lonberg and Huszar, Intern. Rev. Immunol. 13, 65-93 (1995).

In a preferred embodiment of the present invention, the anti-Glutamate receptor antibody is a rat polyclonal anti-GluR3B subregion antibody, directed against an extracellular domain epitope (AA 372-395) of the receptor.

Similarly, intracellular levels of Glutamate signal transducers may be manipulated by increasing the abundance of Glutamate receptor transcripts available for protein synthesis. This may be accomplished by introducing into target cells expressible polynucleotides operatively coding for Glutamate receptor polypeptides. Delivery of such polynucleotides may be by injection, introduction into the circulation, or introduction into the body cavities by inhalation or insufflation. The expressible polynucleotides may be DNA or RNA sequences encoding a Glutamate receptor molecule, capable of enhancing Glutamate stimulation of target cells. Expression may be transient and reversible, or the polynucleotide may become integrated into the host genome, producing stable expression of the therapeutic polynucleotide. For illustrative methodology relating to the introduction of DNA and RNA sequences into host cells, see, for example, U.S. Pat. Nos. 5,589,466 and 6,214,806, both to Felgner et al.

Thus, according to one aspect of the present invention there is provided a method of upregulating T-cell activity in a mammalian subject, the method comprising introducing into at least one T cell related tissue an expressible polynucleotide encoding a Glutamate receptor, the expressible polynucleotide being capable of enhancing expression of Glutamate receptor in the T-cells, thereby upregulating T-cell activity in the mammalian subject. The expressible polynucleotides may contain human Glutamate receptor sequences, at least 60%, preferably at least 70% more preferably at least 80%, more preferably at least 90% and most preferably at least 100% homologous to SEQ ID NOs.1 and 2. Introduction of the expressible polynucleotide may be performed in vivo, or ex vivo, as described in the abovementioned embodiments.

As used herein, the phrase “T cell related tissue” is defined as T cells or T cell progenitors, totipotent cells or any lymphoid tissue being capable of developing into T cells.

Immune deficient conditions that may be treated by the method of the present invention include primary immunodeficiencies, such as the acquired immunodeficiency syndrome (AIDS), DeGeorge's syndrome, reticular dysgenesis, Wiskott/Aldrich syndrome, ataxia-telangiectasia, severe combined immunodeficiency; and secondary immunodeficiencies, such as anergy from tuberculosis, drug-induced leukopenia, non-HIV viral illnesses leukopenia, radiation poisoning, toxin exposure, malnutrition, and the like. Similarly, neoplastic disease or conditions resulting from failure of immune surveillance, and bacterial, fungal and viral infections, especially of the CNS, brain-related injury, degeneration and psychopathology may be treated by upregulation of T-cell function by Glutamate and/or agonist Glutamate analogs or upregulating anti-Glutamate receptor antibodies.

In the context of the present invention, it is important to note the contribution of immune system dysfunction to aging processes. Altered signal transduction and aberrant cytokine production has been demonstrated in T-cells of elderly individuals, and aging T-cells are more susceptible to apoptosis (Pawelec, G. and Solana, R. Immunoageing—the cause or effect of morbidity? Trends in Immunol. 2001: July 22(7) 348-9). Thus, upregulation of T-cell function by Glutamate, upregulating anti-Glutamate receptor antibodies, Glutamate receptor DNA therapy and/or agonist Glutamate analogs may be used to treat immune-related symptoms and processes of aging.

Thus, according to the present invention there is provided a method of relieving or preventing a T-cell related disease or condition in a mammalian subject, the method effected by administering to the subject a therapeutically effective amount of Glutamate or an upregulating Glutamate analog, thereby alleviating the T-cell related disease or condition.

Further, according to the present invention, there is provided a pharmaceutical composition comprising as an active ingredient Glutamate or at least one upregulating Glutamate analog, being packaged and indicated for use in the prevention and/or treatment of an immune deficient condition, in which stimulating T-cell activity is an effective therapy.

Application of the pharmaceutical composition of the present invention may be combined with other therapies and or treatments, wherein the combined application is not contraindicated for either therapy. In one preferred embodiment, the pharmaceutical composition of the present invention is used in combination with T-cell upregulating cytokines. In another embodiment, the pharmaceutical composition of the present invention may be combined with anti-cancer therapy (e.g. radiotherapy, chemotherapy, surgery, dietary therapy, etc) to boost T-cell activity and immune surveillance.

In addition, according to the present invention there is provided an assay of determining an effect of Glutamate or a Glutamate analog on a T-cell related disease or condition, the assay effected by exposing an organism having the aforementioned T-cell related disease or condition to the Glutamate or Glutamate analog and assessing at least one T-cell related symptom of the disease in that organism.

Further, according to the present invention there is provided an assay of determining the sensitivity of a T-cell to Glutamate or a Glutamate analog, the assay effected by exposing the T-cell to one or more concentrations of Glutamate or a Glutamate analog, and assessing a T-cell stimulatory state. In a preferred embodiment the Glutamate or Glutamate analog concentration may be 0.1 ng/ml to 1 mg/ml, sufficient to produce a significant alteration in T-cell activity as measured by, for example, cytokine secretion, adhesion assay, in vitro migration, specific gene expression and the like (see Examples section that follows).

Similarly, the assay of the present invention may be applied to additional methods of upregulating T-cell activity. Thus, the sensitivity of a T-cell to upregulating Glutamate analogs, or to expressible polynucleotides encoding Glutamate receptors and/or to upregulating anti-Glutamate receptor antibodies may be assayed. Exposure of the T-cells to the upregulating modulators may be performed in vivo or in vitro, as described in the Examples section that follows. The expressible polynucleotides may be capable of transient or stable expression in the T-cell. Likewise, the effect of the abovementioned upregulating modulators may be assayed in an organism suffering from an immune deficiency, infectious, age-related or other disease or condition requiring enhanced T-cell activity (see abovementioned recitation of conditions).

As used herein, the term “Glutamate analog” refers to an amino acid, amino acid derivative or other molecule, having a substantial degree of structural or functional identity to Glutamate, being capable of mimicking or modulating Glutamate receptor binding, activation and/or additional steps in Glutamatergic signal transduction pathways. “Agonist analog” refers to analogs causing increased activity of a Glutamate-mediated pathway or target cell function. “Antagonist analog” refers to analogs inhibiting, or reducing activity in a Glutamate mediated pathway or target cell function. Exhaustive lists of agonist and antagonist Glutamate analogs are available to one skilled in the art (see Tocris Neurochemicals, Section 3, page 24-35, TOCRIS, UK; and RBI Catalog, pg 401-03, Sigma-RBI, St. Louis, Mo.).

As used herein, the term “naturally-occurring” as applied to an object refers to the fact that an object can be found in nature. For example, an amino acid, polypeptide or polynucleotide sequence that is present in an organism (including viruses) that can be isolated from a source in and which has not been intentionally modified by man in the laboratory is naturally-occurring.

Accordingly, as used herein the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Diseases or conditions requiring suppression of immune function may be sensitive to inhibition of T-cell activity by antagonist Glutamate analogs, downregulating anti-Glutamate receptor antibodies, and/or polynucleotides downregulating Glutamate receptor expression. These diseases or conditions include autoimmune and hyperreactive states such as systemic lupus erythematosis, rheumatic fever, rheumatoid arthritis, multiple sclerosis, Hashimoto's and Grave's disease, Goodpasture's syndrome, myasthenia gravis, insulin-dependent diabetes mellitus, pemphigus vulgaris, Addison's disease, dermatitis herpetiformis and celiac disease; allergic conditions such as atopic dermatitis, allergic asthma, anaphylaxis and other IgE-mediated responses. Similarly, other conditions of undesired T-cell migration and function include T-cell cancer such as T-lymphoma and other myeloproliferative diseases, T-cell mediated graft versus host disease, allograft rejection, neuronal damage and psychopathology.

As used herein, the term “psychopathology” refers to any and all disease, disorder, syndrome, etc. characterized by mental or emotional dysfunction, mental illness or social dysorganization. Some non-limiting examples of such disorders are affective disorders, bipolar disorders, obsessive-compulsive disorders, anxiety disorders, phobias, post traumatic stress disorder, psychogeriatric disorders, somatoform disorders, personality disorders, multiple personality disorders, schizophrenia, autism, psychoneuroses and psychoses (see Handbook of Psychiatric Drugs, S. E. Hyman ed., Little, Brown and Co., Boston 1995).

While reducing the present invention to practice, it was demonstrated that Glutamate modulation of T-cell function was mediated by the neurotransmitter's effect on T-cell binding to fibronectin and chemokine-mediated cell migration. Importantly, substrate recognition and binding of T-cell surface adhesion molecules (e.g. integrins) is an essential step in T-lymphoma extravasion and metastasis (Bittner M, et al J Immunol 1998; 161:5978-86; Wang J M et al Int J Cancer 1998; 75:900-7 and Wilson K E et al J Immunol 1999; 163:3621-28). Therefore, inhibition of T-cell binding and chemokine-mediated migration by antagonist Glutamate analogs, downregulating anti-Glutamate receptor antibodies, and/or polynucleotides downregulating Glutamate receptor expression may be effective in preventing and/or treating T-cell binding and migration-related neoplastic and metastatic conditions.

Thus, according to the present invention there is provided a method of modulating T-cell activity, the method effected by exposing T-cells to a downregulating Glutamate analog, causing decreased T-cell activity. The Glutamate downregulating analog may be naturally occurring or synthetic. In one embodiment, the downregulator is CNQX. In a preferred embodiment, the downregulator is a Glutamate receptor blocker. In a more preferred embodiment, the downregulating receptor blocker is an anti-Glutamate receptor antibody. In a most preferred embodiment the downregulator is a GluR3 specific antagonist Glutamate analog. Such downregulating Glutamate antagonist analogs are readily available to one skilled in the art (see abovementioned catalog references).

As mentioned above, T-cells may be isolated from the blood by procedures known to one skilled in the art (see, for example, the Materials and Methods section that follows). Thus, in the method of the present invention T-cells may be exposed to the downregulating analog or anti-Glutamate receptor antibody in vivo, by administration to the subject via intravenous, parenteral, oral, transdermal, intramuscular, intranasal or other means or in vitro, after removal of T-cells from the body and their isolation.

Further according to the present invention there is provided a method of downregulating T-cell activity in a mammalian subject, the method effected by administering to the subject a therapeutically effective amount of a T-cell downregulating Glutamate analog thereby downregulating T-cell activity in the mammalian subject. In one embodiment, the downregulator is a Glutamate receptor blocker. In an additional embodiment, the downregulating Glutamate analog is a downregulator of T-cell adhesion and migration. In another, preferred embodiment the antagonist Glutamate analog may be a naturally occurring or synthetic analog. In the method of the present invention T-cells may be exposed to the downregulating analog in vivo, by administration to the subject via intravenous, parenteral, oral, transdermal, intramuscular, intranasal or other means or in vitro, after removal of T-cells from the body and their isolation.

Intracellular levels of Glutamate signal transducers may be manipulated by decreasing the abundance of Glutamate receptor transcripts available for protein synthesis. This may be accomplished by introducing into target cells polynucleotides downregulating Glutamate receptor expression. Delivery of such polynucleotides may be by injection, introduction into the circulation, or introduction into the body cavities by inhalation or insufflation.

Thus, according to the present invention, there is provided a method of downregulating T-cell activity in a mammalian subject, the method effected by introducing into T-cells of the subject a polynucleotide which down-regulates Glutamate receptor expression, the polynucleotide being capable of reducing Glutamate receptor expression in the cells, effectively reducing sensitivity to Glutamate activation, thereby downregulating T-cell activity in the mammalian subject. The polynucleotides may be ribozymes having specific Glutamate receptor transcript cleaving capability, or antisense nucleotide sequences complementary to, and capable of binding to Glutamate receptor transcripts, coding sequences and/or promoter elements. These polynucleotide sequences may be introduced into the organisms T-cells and other tissues in vivo or in vitro, as described in the aforementioned embodiments, according to the principles and techniques recited hereinbelow.

An antisense polynucleotide (e.g., antisense oligodeoxyribonucleotide) may bind its target nucleic acid either by Watson-Crick base pairing or Hoogsteen and anti-Hoogsteen base pairing (Thuong and Helene (1993) Sequence specific recognition and modification of double helical DNA by oligonucleotides Angev. Chem. Int. Ed. Engl. 32:666). According to the Watson-Crick base pairing, heterocyclic bases of the antisense polynucleotide form hydrogen bonds with the heterocyclic bases of target single-stranded nucleic acids (RNA or single-stranded DNA), whereas according to the Hoogsteen base pairing, the heterocyclic bases of the target nucleic acid are double-stranded DNA, wherein a third strand is accommodated in the major groove of the B-form DNA duplex by Hoogsteen and anti-Hoogsteen base pairing to form a triple helix structure.

According to both the Watson-Crick and the Hoogsteen base pairing models, antisense oligonucleotides have the potential to regulate gene expression and to disrupt the essential functions of the nucleic acids in cells. Therefore, antisense polynucleotides have possible uses in modulating a wide range of diseases in which gene expression is altered.

Since the development of effective methods for chemically synthesizing polynucleotides, these molecules have been extensively used in biochemistry and biological research and have the potential use in medicine, since carefully devised polynucleotides can be used to control gene expression by regulating levels of transcription, transcripts and/or translation.

Oligodeoxyribonucleotides as long as 100 base pairs (bp) are routinely synthesized by solid phase methods using commercially available, fully automated synthesis machines. The chemical synthesis of oligoribonucleotides, however, is far less routine. Oligoribonucleotides are also much less stable than oligodeoxyribonucleotides, a fact which has contributed to the more prevalent use of oligodeoxyribonucleotides in medical and biological research, directed at, for example, the regulation of transcription or translation levels.

Gene expression involves few distinct and well regulated steps. The first major step of gene expression involves transcription of a messenger RNA (mRNA) which is an RNA sequence complementary to the antisense (i.e., −) DNA strand, or, in other words, identical in sequence to the DNA sense (i.e., +) strand, composing the gene. In eukaryotes, transcription occurs in the cell nucleus.

The second major step of gene expression involves translation of a protein (e.g., enzymes, structural proteins, secreted proteins, gene expression factors, etc.) in which the mRNA interacts with ribosomal RNA complexes (ribosomes) and amino acid activated transfer RNAs (tRNAs) to direct the synthesis of the protein coded for by the mRNA sequence.

Initiation of transcription requires specific recognition of a promoter DNA sequence located upstream to the coding sequence of a gene by an RNA-synthesizing enzyme—RNA polymerase. This recognition is preceded by sequence-specific binding of one or more transcription factors to the promoter sequence. Additional proteins which bind at or close to the promoter sequence may trans upregulate transcription via cis elements known as enhancer sequences. Other proteins which bind to or close to the promoter, but whose binding prohibits the action of RNA polymerase, are known as repressors.

There is also evidence that in some cases gene expression is downregulated by endogenous antisense RNA repressors that bind a complementary mRNA transcript and thereby prevent its translation into a functional protein.

Thus, gene expression is typically upregulated by transcription factors and enhancers and downregulated by repressors.

However, in many disease situations gene expression is impaired. In many cases, such as different types of cancer, for various reasons the expression of a specific endogenous or exogenous (e.g., of a pathogen such as a virus) gene is upregulated.

The ability to chemically synthesize oligonucleotides and analogs thereof having a selected predetermined sequence offers means for downmodulating gene expression. Three types of gene expression modulation strategies may be considered.

At the transcription level, antisense or sense oligonucleotides or analogs that bind to the genomic DNA by strand displacement or the formation of a triple helix, may prevent transcription (Thuong and Helene (1993) Sequence specific recognition and modification of double helical DNA by oligonucleotides Angev. Chem. Int. Ed. Engl. 32:666).

At the transcript level, antisense oligonucleotides or analogs that bind target mRNA molecules lead to the enzymatic cleavage of the hybrid by intracellular RNase hours (Dash P., Lotan I., Knapp M., Kandel E. R. and Goelet P. (1987) Selective elimination of mRNAs in vivo: complementary oligodeoxynucleotides promote RNA degradation by an RNase H-like activity. Proc. Natl. Acad. Sci. USA, 84:7896). In this case, by hybridizing to the targeted mRNA, the oligonucleotides or oligonucleotide analogs provide a duplex hybrid recognized and destroyed by the RNase hours enzyme. Alternatively, such hybrid formation may lead to interference with correct splicing (Chiang M. Y., Chan H., Zounes M. A., Freier S. M., Lima W. F. and Bennett C. F. (1991) Antisense oligonucleotides inhibit intercellular adhesion molecule 1 expression by two distinct mechanisms. J. Biol. Chem. 266:18162-71). As a result, in both cases, the number of the target mRNA intact transcripts ready for translation is reduced or eliminated.

At the translation level, antisense oligonucleotides or analogs that bind target mRNA molecules prevent, by steric hindrance, binding of essential translation factors (ribosomes), to the target mRNA, a phenomenon known in the art as hybridization arrest, disabling the translation of such mRNAs.

Thus, antisense sequences, which as described hereinabove may arrest the expression of any endogenous and/or exogenous gene depending on their specific sequence, attracted much attention by scientists and pharmacologists who were devoted at developing the antisense approach into a new pharmacological tool.

For example, several antisense oligonucleotides have been shown to arrest hematopoietic cell proliferation (Szczylik et al. (1991) Selective inhibition of leukemia cell proliferation by BCR-ABL antisense oligodeoxynucleotides. Science 253:562.), growth (Calabretta et al. (1991) Normal and leukemic hematopoietic cell manifest differential sensitivity to inhibitory effects of c-myc antisense oligodeoxynucleotides: an in vitro study relevant to bone marrow purging. Proc. Natl. Acad. Sci. USA 88:2351), entry into the S phase of the cell cycle (Heikhila et al. (1987) A c-myc antisense oligodeoxynucleotide inhibits entry into S phase but not progress from G(0) to G(1). Nature, 328:445), reduced survival (Reed et al. (1990) Antisense mediated inhibition of BCL2 prooncogene expression and leukemic cell growth and survival: comparison of phosphodiester and phosphorothioate oligodeoxynucleotides. Cancer Res. 50:6565), prevent receptor mediated responses (Burch and Mahan (1991) Oligodeoxynucleotides antisense to the interleukin I receptor m RNA block the effects of interleukin I in cultured murine and human fibroblasts and in mice. J. Clin. Invest. 88:1190) and as antiviral agents (Agrawal (1992) Antisense oligonucleotides as antiviral agents. TIBTECH 10:152).

For efficient in vivo inhibition of gene expression using antisense oligonucleotides or analogs, the oligonucleotides or analogs must fulfill the following requirements (i) sufficient specificity in binding to the target sequence; (ii) solubility in water; (iii) stability against intra- and extracellular nucleases; (iv) capability of penetration through the cell membrane; and (v) when used to treat an organism, low toxicity.

Unmodified oligonucleotides are impractical for use as antisense sequences since they have short in vivo half-lives, during which they are degraded rapidly by nucleases. Furthermore, they are difficult to prepare in more than milligram quantities. In addition, such oligonucleotides are poor cell membrane penetrators.

Thus it is apparent that in order to meet all the above listed requirements, oligonucleotide analogs need to be devised in a suitable manner. Therefore, an extensive search for modified oligonucleotides has been initiated.

For example, problems arising in connection with double-stranded DNA (dsDNA) recognition through triple helix formation have been diminished by a clever “switch back” chemical linking, whereby a sequence of polypurine on one strand is recognized, and by “switching back”, a homopurine sequence on the other strand can be recognized. Also, good helix formation has been obtained by using artificial bases, thereby improving binding conditions with regard to ionic strength and pH.

In addition, in order to improve half-life as well as membrane penetration, a large number of variations in polynucleotide backbones have been done, nevertheless with little success.

Oligonucleotides can be modified either in the base, the sugar or the phosphate moiety. These modifications include, for example, the use of methylphosphonates, monothiophosphates, dithiophosphates, phosphoramidates, phosphate esters, bridged phosphorothioates, bridged phosphoramidates, bridged methylenephosphonates, dephospho internucleotide analogs with siloxane bridges, carbonate bridges, carboxymethyl ester bridges, carbonate bridges, carboxymethyl ester bridges, acetamide bridges, carbamate bridges, thioether bridges, sulfoxy bridges, sulfono bridges, various “plastic” DNAs, α-anomeric bridges and borane derivatives. For illustrative examples and further details see Cook (1991) Medicinal chemistry of antisense oligonucleotides—future opportunities. Anti-Cancer Drug Design 6:585.

International patent application WO 89/12060 discloses various building blocks for synthesizing oligonucleotide analogs, as well as oligonucleotide analogs formed by joining such building blocks in a defined sequence. The building blocks may be either “rigid” (i.e., containing a ring structure) or “flexible” (i.e., lacking a ring structure). In both cases, the building blocks contain a hydroxy group and a mercapto group, through which the building blocks are said to join to form oligonucleotide analogs. The linking moiety in the oligonucleotide analogs is selected from the group consisting of sulfide (—S—), sulfoxide (—SO—), and sulfone (—SO₂—). However, the application provides no data supporting the specific binding of an oligonucleotide analog to a target oligonucleotide.

International patent application WO 92/20702 describe an acyclic oligonucleotide which includes a peptide backbone on which any selected chemical nucleobases or analogs are stringed and serve as coding characters as they do in natural DNA or RNA. These new compounds, known as peptide nucleic acids (PNAs), are not only more stable in cells than their natural counterparts, but also bind natural DNA and RNA 50 to 100 times more tightly than the natural nucleic acids cling to each other. PNA oligomers can be synthesized from the four protected monomers containing thymine, cytosine, adenine and guanine by Merrifield solid-phase peptide synthesis. In order to increase solubility in water and to prevent aggregation, a lysine amide group is placed at the C-terminal.

Thus, antisense technology requires pairing of messenger RNA with an oligonucleotide to form a double helix that inhibits translation. The concept of antisense-mediated gene therapy was already introduced in 1978 for cancer therapy. This approach was based on certain genes that are crucial in cell division and growth of cancer cells. Synthetic fragments of genetic substance DNA can achieve this goal. Such molecules bind to the targeted gene molecules in RNA of tumor cells, thereby inhibiting the translation of the genes and resulting in dysfunctional growth of these cells. Other mechanisms has also been proposed. These strategies have been used, with some success in treatment of cancers, as well as other illnesses, including viral and other infectious diseases. Antisense polynucleotides are typically synthesized in lengths of 13-30 nucleotides. The life span of oligonucleotide molecules in blood is rather short. Thus, they have to be chemically modified to prevent destruction by ubiquitous nucleases present in the body. Phosphorothioates are very widely used in modifying antisense oligonucleotide in ongoing clinical trials. A new generation of antisense molecules consists of hybrid antisense oligonucleotides with a central portion of synthetic DNA while four bases on each end have been modified with 2′O-methyl ribose to resemble RNA. In preclinical studies in laboratory animals, such compounds have demonstrated greater stability to metabolism in body tissues and an improved safety profile when compared with the first-generation unmodified phosphorothioate (Hybridon Inc. News). Dozens of other nucleotide analogs have also been tested in antisense technology.

RNA oligonucleotides may also be used for antisense inhibition as they form a stable RNA-RNA duplex with the target, suggesting efficient inhibition. However, due to their low stability RNA oligonucleotides are typically expressed inside the cells using vectors designed for this purpose. This approach is favored when attempting to target a mRNA that encodes an abundant and long-lived protein.

Recent scientific publications have validated the efficacy of antisense compounds in animal models of hepatitis, cancers, coronary artery restenosis and other diseases. The first antisense drug was recently approved by the FDA. This drug Fomivirsen, developed by Isis, is indicated for local treatment of cytomegalovirus in patients with AIDS who are intolerant of or have a contraindication to other treatments for CMV retinitis or who were insufficiently responsive to previous treatments for CMV retinitis (Pharmacotherapy News Network).

Several antisense compounds are now in clinical trials in the United States. These include locally administered antivirals, systemic cancer therapeutics. Antisense therapeutics has the potential to treat many life-threatening diseases with a number of advantages over traditional drugs. Traditional drugs intervene after a disease-causing protein is formed. Antisense therapeutics, however, block mRNA transcription/translation and intervene before a protein is formed, and since antisense therapeutics target only one specific mRNA, they should be more effective with fewer side effects than current protein-inhibiting therapy.

Antisense therapy has also been applied to immune disorders and inhibition of cell migration. For example, U.S. Pat. No. 6,096,722 to Bennet et al. discloses the application of antisense polynucleotides to interrupt cell adhesion molecules (CAM) expression in the treatment of pathogenic, autoimmune, allergic, chronic inflammatory, hyperproliferation and metastatic conditions.

A second option for disrupting gene expression at the level of transcription uses synthetic oligonucleotides capable of hybridizing with double stranded DNA. A triple helix is formed. Such oligonucleotides may prevent binding of transcription factors to the gene's promoter and therefore inhibit transcription. Alternatively, they may prevent duplex unwinding and, therefore, transcription of genes within the triple helical structure.

Another approach is the use of specific nucleic acid sequences to act as decoys for transcription factors. Since transcription factors bind specific DNA sequences it is possible to synthesize oligonucleotides that will effectively compete with the native DNA sequences for available transcription factors in vivo. This approach requires the identification of gene specific transcription factor.

Indirect inhibition of gene expression was demonstrated for matrix metalloproteinase genes (MMP-1, -3, and -9), which are associated with invasive potential of human cancer cells. E1AF is a transcription activator of MMP genes. Expression of E1AF antisense RNA in HSC3AS cells showed decrease in mRNA and protein levels of MMP-1, -3, and -9. Moreover, HSC3AS showed lower invasive potential in vitro and in vivo. These results imply that transfection of antisense inhibits tumor invasion by down-regulating MMP genes.

Ribozymes are being increasingly used for the sequence-specific inhibition of gene expression by the cleavage of mRNAs encoding proteins of interest. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pha, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page).

As used herein, “ribozymes” are intended to include RNA molecules that contain anti-sense sequences for specific recognition, and an RNA-cleaving enzymatic activity. The catalytic strand cleaves a specific site in a target RNA at greater than stoichiometric concentration. Two “types” of ribozymes are particularly useful in this invention, the hammerhead ribozyme (Rossi, J. J. et al., Pharmac. Ther. 50:245-254, 1991) and the hairpin ribozyme (Hampel et al., Nucl. Acids Res. 18:299-304, 1990, and U.S. Pat. No. 5,254,678, issued Oct. 19, 1993). Because both hammerhead and hairpin ribozymes are catalytic molecules having antisense and endoribonucleotidase activity, ribozyme technology has emerged as a potential powerful extension of the antisense approach to gene inactivation.

The ribozymes of the invention typically consist of RNA, but such ribozymes may also be composed of nucleic acid molecules comprising chimeric nucleic acid sequences (such as DNA/RNA sequences) and/or nucleic acid analogs (e.g., phosphorothioates). Ribozymes may be in the form of a “hammerhead” (for example, as described by Forster and Symons, Cell 48:211-220, 1987; Haseloff and Gerlach, Nature 328:596-600, 1988; Walbot and Bruening, Nature 334:196, 1988; Haseloff and Gerlach, Nature 334:585, 1988) or a “hairpin” (for example, as described by Haseloff et al., U.S. Pat. No. 5,254,678, issued Oct. 19, 1993 and Hempel et al., European Patent Publication No. 0 360 257, published Mar. 26, 1990). The sequence requirement for the hairpin ribozyme is any RNA sequence consisting of NNNBN*GUCNNNNNN (where N*G is the cleavage site, where B is any of G, C, or U, and where N is any of G, U, C, or A) (SEQ ID NO:3). The sequence requirement at the cleavage site for the hammerhead ribozyme is any RNA sequence consisting of NUX (where N is any of G, U, C, or A and X represents C, U, or A) can be targeted. Accordingly, the same target within the hairpin leader sequence, GUC, is useful for the hammerhead ribozyme. The additional nucleotides of the hammerhead ribozyme or hairpin ribozyme is determined by the target flanking nucleotides and the hammerhead consensus sequence (see Ruffner et al., Biochemistry 29:10695-10702, 1990).

This information, and the published mRNA sequences of human Glutamate receptor GluR3 flop isoform (Genbank accession number U10302; Kamboj, R K et al.) (SEQ ID NO:1) and human Glutamate receptor GluR3 flip isoform (Genbank accession number U10301; Kamboj R K) (SEQ ID NO:2) together with the genomic and cDNA sequences for other Glutamate receptor genes enables production of the ribozymes of this invention. Appropriate base changes in the ribozyme are made to maintain the necessary base pairing with the target RNA sequences.

Cech et al. (U.S. Pat. No. 4,987,071) has disclosed the preparation and use of certain synthetic ribozymes which have endoribonuclease activity. These ribozymes are based on the properties of the Tetrahymena ribosomal RNA self-splicing reaction and require an eight base pair target site. The ribozymes of this invention, as well as DNA encoding such ribozymes and other suitable nucleic acid molecules, can be chemically synthesized using methods well known in the art for the synthesis of nucleic acid molecules. Alternatively, Promega, Madison, Wis., USA, provides a series of protocols suitable for the production of RNA molecules such as ribozymes. The ribozymes also can be prepared from a DNA molecule or other nucleic acid molecule (which, upon transcription, yields an RNA molecule) operably linked to an RNA polymerase promoter, e.g., the promoter for T7 RNA polymerase or SP6 RNA polymerase. Such a construct may be referred to as a vector. Accordingly, also provided by this invention are nucleic acid molecules, e.g., DNA or cDNA, coding for the ribozymes of this invention. When the vector also contains an RNA polymerase promoter operably linked to the DNA molecule, the ribozyme can be produced in vitro upon incubation with the RNA polymerase and appropriate nucleotides. Alternatively, the DNA may be inserted into an expression cassette, such as described in Cotten and Birnstiel, EMBO J. 8(12):3861-3866, 1989, and in Hempel et al., Biochemistry 28:4929-4933, 1989. A more detailed discussion of molecular biology methodology is disclosed in Sambrook et al., Molecular Cloning: A Laboratory Manual, Cold Spring Harbor Press, 1989.

After synthesis, the ribozyme can be modified by ligation to a DNA molecule having the ability to stabilize the ribozyme and make it resistant to RNase. Alternatively, the ribozyme can be modified to the phosphothio analog for use in liposome delivery systems. This modification also renders the ribozyme resistant to endonuclease activity.

The ribozyme molecule also can be in a host prokaryotic or eukaryotic cell in culture or in the cells of an organism. Appropriate prokaryotic and eukaryotic cells can be transfected with an appropriate transfer vector containing the DNA molecule encoding a ribozyme of this invention. Alternatively, the ribozyme molecule, including nucleic acid molecules encoding the ribozyme, may be introduced into the host cell using traditional methods such as transformation using calcium phosphate precipitation (Dubensky et al., PNAS 81:7529-7533, 1984), direct microinjection of such nucleic acid molecules into intact target cells (Acsadi et al., Nature 352:815-818, 1991), and electroporation whereby cells suspended in a conducting solution are subjected to an intense electric field in order to transiently polarize the membrane, allowing entry of the nucleic acid molecules. Other procedures include the use of nucleic acid molecules linked to an inactive adenovirus (Cotton et al., PNAS 89:6094, 1990), lipofection (Felgner et al., Proc. Natl. Acad. Sci. USA 84:7413-7417, 1989), microprojectile bombardment (Williams et al., PNAS 88:2726-2730, 1991), polycation compounds such as polylysine, receptor specific ligands, liposomes entrapping the nucleic acid molecules, spheroplast fusion whereby E coli containing the nucleic acid molecules are stripped of their outer cell walls and fused to animal cells using polyethylene glycol, viral transduction, (Cline et al., Pharmac. Ther. 29:69, 1985; and Friedmann et al., Science 244:1275, 1989), and DNA ligand (Wu et al. J. of Biol. Chem. 264:16985-16987, 1989), as well as psoralen inactivated viruses such as Sendai or Adenovirus. In a preferred embodiment, the ribozyme is introduced into the host cell utilizing a liposome.

When the DNA molecule is operatively linked to a promoter for RNA transcription, the RNA can be produced in the host cell when the host cell is grown under suitable conditions favoring transcription of the DNA molecule. The vector can be, but is not limited to a plasmid, a virus, a retrotransposon or a cosmid. Examples of such vectors are disclosed in U.S. Pat. No. 5,166,320. Other representative vectors include adenoviral vectors (e.g., WO 94/26914, WO 93/9191; Kolls et al., PNAS 91(1):215-219, 1994; Kass-Eisler et al., PNAS 90(24):11498-502, 1993; Guzman et al., Circulation 88(6):2838-48, 1993; Guzman et al., Cir. Res. 73(6):1202-1207, 1993; Zabner et al., Cell 75(2):207-216, 1993; Li et al., Huim Gene Ther. 4(4):403-409, 1993; Caillaud et al., Eur. J. Neurosci. 5(10):1287-1291, 1993), adeno-associated vector type 1 (“AAV-1”) or adeno-associated vector type 2 (“AAV-2”) (see WO 95/13365; Flotte et al., PNAS 90(22):10613-10617, 1993), retroviral vectors (e.g., EP 0 415 731; WO 90/07936; WO 91/02805; WO 94/03622; WO 93/25698; WO 93/25234; U.S. Pat. No. 5,219,740; WO 93/11230; WO 93/10218) and herpes viral vectors (e.g., U.S. Pat. No. 5,288,641). Methods of utilizing such vectors in gene therapy are well known in the art, see, for example, Larrick, J. W. and B, K. L., Gene Therapy: Application of Molecular Biology, Elsevier Science Publishing Co., Inc., New York, N.Y., 1991 and Kreigler, M., Gene Transfer and Expression: A Laboratory Manual, W.H. Freeman and Company, New York, 1990. To produce ribozymes in vivo utilizing vectors, the nucleotide sequences coding for ribozymes are preferably placed under the control of a strong promoter such as the lac, SV40 late, SV40 early, or lambda promoters. Ribozymes are then produced directly from the transfer vector in vivo.

Observations in the early 1990s that plasmid DNA could directly transfect animal cells in vivo sparked exploration of the use of DNA plasmids to induce immune response by direct injection into animal of DNA encoding antigenic protein. When a DNA vaccine plasmid enters the eukaryotic cell, the protein it encodes is transcribed and translated within the cell. In the case of pathogens, these proteins are presented to the immune system in their native form, mimicking the presentation of antigens during a natural infection. DNA vaccination is particularly useful for the induction of T cell activation. It was applied for viral and bacterial infectious diseases, as well as for allergy and for cancer. The central hypothesis behind active specific immunotherapy for cancer is that tumor cells express unique antigens that should stimulate the immune system. The first DNA vaccine against tumor was carcino-embrionic antigen (CEA). DNA vaccinated animals expressed immunoprotection and immunotherapy of human CEA-expressing syngeneic mouse colon and breast carcinoma. In a mouse model of neuroblastoma, DNA immunization with HuD resulted in tumor growth inhibition with no neurological disease. Immunity to the brown locus protein, gp⁷⁵ tyrosinase-related protein-1, associated with melanoma, was investigated in a syngeneic mouse model. Priming with human gp75 DNA broke tolerance to mouse gp75. Immunity against mouse gp75 provided significant tumor protection.

The present invention has the potential to provide transgenic gene and polymorphic gene animal and cellular (cell lines) models as well as for knockout models. These models may be constructed using standard methods known in the art and as set forth in U.S. Pat. Nos. 5,487,992, 5,464,764, 5,387,742, 5,360,735, 5,347,075, 5,298,422, 5,288,846, 5,221,778, 5,175,385, 5,175,384, 5,175,383, 4,736,866 as well as Burke and Olson, Methods in Enzymology, 194:251-270 1991); Capecchi, Science 244:1288-1292 1989); Davies et al., Nucleic Acids Research, 20 (11) 2693-2698 1992); Dickinson et al., Human Molecular Genetics, 2(8): 1299-1302 1993); Duff and Lincoln, “Insertion of a pathogenic mutation into a yeast artificial chromosome containing the human APP gene and expression in ES cells”, Research Advances in Alzheimer's Disease and Related Disorders, 1995; Huxley et al., Genomics, 9:-750 1991); Jakobovits et al., Nature, 362:255-261 1993); Lamb et al., Nature Genetics, 5: 22-29 1993); Pearson and Choi, Proc. Natl. Acad. Sci. USA 1993). 90:10578-82; Rothstein, Methods in Enzymology, 194:281-301 1991); Schedl et al., Nature, 362: 258-261 1993); Strauss et al., Science, 259:1904-1907 1993). Further, patent applications WO 94/23049, WO93/14200, WO 94/06908, WO 94/28123 also provide information.

Gene therapy as used herein refers to the transfer of genetic material (e.g. DNA or RNA) of interest into a host to treat or prevent a genetic or acquired disease or condition or phenotype. The genetic material of interest encodes a product (e.g. a protein, polypeptide, peptide, functional RNA, antisense) whose production in vivo is desired. For example, the genetic material of interest can encode a hormone, receptor, enzyme, polypeptide or peptide of therapeutic value. For review see, in general, the text “Gene Therapy” (Advanced in Pharmacology 40, Academic Press, 1997).

Two basic approaches to gene therapy have evolved: (1) ex vivo and (2) in vivo gene therapy. In ex vivo gene therapy cells are removed from a patient, and while being cultured are treated in vitro. Generally, a functional replacement gene is introduced into the cell via an appropriate gene delivery vehicle/method (transfection, transduction, homologous recombination, etc.) and an expression system as needed and then the modified cells are expanded in culture and returned to the host/patient. These genetically reimplanted cells have been shown to express the transfected genetic material in situ.

In in vivo gene therapy, target cells are not removed from the subject rather the genetic material to be transferred is introduced into the cells of the recipient organism in situ, that is, within the recipient. In an alternative embodiment, if the host gene is defective, the gene is repaired in situ (Culver, 1998. (Abstract) Antisense DNA & RNA based therapeutics, February 1998, Coronado, Calif.).

These genetically altered cells have been shown to express the transfected genetic material in situ.

The gene expression vehicle is capable of delivery/transfer of heterologous nucleic acid into a host cell. The expression vehicle may include elements to control targeting, expression and transcription of the nucleic acid in a cell selective manner as is known in the art. It should be noted that often the 5′UTR and/or 3′UTR of the gene may be replaced by the 5′UTR and/or 3′UTR of the expression vehicle. Therefore, as used herein the expression vehicle may, as needed, not include the 5′UTR and/or 3′UTR of the actual gene to be transferred and only include the specific amino acid coding region.

The expression vehicle can include a promoter for controlling transcription of the heterologous material and can be either a constitutive or inducible promoter to allow selective transcription. Enhancers that may be required to obtain necessary transcription levels can optionally be included. Enhancers are generally any nontranslated DNA sequence which works contiguously with the coding sequence (in cis) to change the basal transcription level dictated by the promoter. The expression vehicle can also include a selection gene as described herein below.

Vectors can be introduced into cells or tissues by any one of a variety of known methods within the art. Such methods can be found generally described in Sambrook et al., Molecular Cloning: A Laboratory Manual, Cold Springs Harbor Laboratory, New York 1989, 1992), in Ausubel et al., Current Protocols in Molecular Biology, John Wiley and Sons, Baltimore, Md. 1989), Chang et al., Somatic Gene Therapy, CRC Press, Ann Arbor, Mich. 1995), Vega et al., Gene Targeting, CRC Press, Ann Arbor Mich. (995), Vectors: A Survey of Molecular Cloning Vectors and Their Uses, Butterworths, Boston Mass. 1988) and Gilboa et al. (Biotechniques 4 (6): 504-512, 1986) and include, for example, stable or transient transfection, lipofection, electroporation and infection with recombinant viral vectors. In addition, see U.S. Pat. No. 4,866,042 for vectors involving the central nervous system and also U.S. Pat. Nos. 5,464,764 and 5,487,992 for positive-negative selection methods.

Introduction of nucleic acids by infection with viral vectors offers several advantages over the other listed methods. Higher efficiency can be obtained due to their infectious nature. Moreover, viruses are very specialized and typically infect and propagate in specific cell types. Thus, their natural specificity can be used to target the vectors to specific cell types in vivo or within a tissue or mixed culture of cells. Viral vectors can also be modified with specific receptors or ligands to alter target specificity through receptor mediated events.

A specific example of DNA viral vector introducing and expressing recombination sequences is the adenovirus-derived vector Adenop53TK. This vector expresses a herpes virus thymidine kinase (TK) gene for either positive or negative selection and an expression cassette for desired recombinant sequences. This vector can be used to infect cells that have an adenovirus receptor which includes most cancers of epithelial origin as well as others. This vector as well as others that exhibit similar desired functions can be used to treat a mixed population of cells and can include, for example, an in vitro or ex vivo culture of cells, a tissue or a human subject.

Features that limit expression to particular cell types can also be included. Such features include, for example, promoter and regulatory elements that are specific for the desired cell type.

In addition, recombinant viral vectors are useful for in vivo expression of a desired nucleic acid because they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

As described above, viruses are very specialized infectious agents that have evolved, in many cases, to elude host defense mechanisms. Typically, viruses infect and propagate in specific cell types. The targeting specificity of viral utilizes its natural specificity of viral vectors utilizes its natural specificity to specifically target predetermined cell types and thereby introduce a recombinant gene into the infected cell. The vector to be used in the methods of the invention will depend on desired cell type to be targeted and will be known to those skilled in the art. For example, if breast cancer is to be treated then a vector specific for such epithelial cells would be used. Likewise, if diseases or pathological conditions of the hematopoietic system are to be treated, then a viral vector that is specific for blood cells and their precursors, preferably for the specific type of hematopoietic cell, would be used.

Retroviral vectors can be constructed to function either as infectious particles or to undergo only a single initial round of infection. In the former case, the genome of the virus is modified so that it maintains all the necessary genes, regulatory sequences and packaging signals to synthesize new viral proteins and RNA. Once these molecules are synthesized, the host cell packages the RNA into new viral particles which are capable of undergoing further rounds of infection. The vector's genome is also engineered to encode and express the desired recombinant gene. In the case of non-infectious viral vectors, the vector genome is usually mutated to destroy the viral packaging signal that is required to encapsulate the RNA in viral particles. Without such signal, any particles that are formed will not contain a genome and therefore cannot proceed through subsequent rounds of infection. The specific type of vector will depend upon the intended application. The actual vectors are also known and readily available within the art or can be constructed by one skilled in the art using well-known methodology.

The recombinant vector can be administered in several ways. If viral vectors are used, for example, the procedure can take advantage of their target specificity and consequently, do not have to be administered locally at the diseased site. However, local administration can provide a quicker and more effective treatment, administration can also be performed by, for example, intravenous or subcutaneous injection into the subject. Injection of the viral vectors into a spinal fluid can also be used as a mode of administration, especially in the case of neuro-degenerative diseases. Following injection, the viral vectors will circulate until they recognize host cells with appropriate target specificity for infection.

Thus, according to yet another aspect of the present invention there is provided a method of downregulating T-cell activity in a mammalian subject, the method comprising introducing into the cells an expressible polynucleotide that downregulates Glutamate receptor expression, the expressible polynucleotide being capable of reducing expression of a Glutamate receptor, effectively reducing sensitivity to Glutamate stimulation, thereby downregulating T-cell activity in the mammalian subject. The expressible polynucleotides may contain sequences at least 60%, preferably at least 70%, more preferably at least 80%, more preferably at least 90% and most preferably about 100% complementary to SEQ ID NOs:1 and 2. In one preferred embodiment, the polynucleotide is a ribozyme having specific Glutamate receptor transcript cleaving capability. In another preferred embodiment, the polynucleotide is an expressible polynucleotide encoding a ribozyme having specific Glutamate receptor transcript cleaving capability.

In yet another embodiment of the present invention, the polynucleotide is an antisense oligonucleotide, comprising nucleotide sequences complementary to, and capable of binding to Glutamate receptor transcripts, coding sequences and/or promoter elements. As described above, antisense inhibition of expression of a Glutamate receptor may be achieved by DNA therapy. For example, Baserga et al. (U.S. Pat. No. 6,274,562) discloses the application of antisense constructs against IGF-I receptor transcripts to inhibit proliferation and cause differentiation of the IGF-I sensitive cells. Schreiber et al. (U.S. Pat. No. 6,242,427) disclose antisense constructs for treatment of inflammatory conditions by inhibiting Fc receptor expression in phagocytic cells. Thus, in another preferred embodiment, the down-regulating polynucleotide is an expressible polynucleotide encoding sequences complementary to, and capable of binding to Glutamate receptor transcripts, coding sequences and/or promoter elements.

In a preferred embodiment, downregulation of T-cell activity by ribozyme, antisense or DNA methodology directed against a Glutamate receptor is applied where the mammalian subject is suffering from immune hyperfunction or hyperreactivity, such as in autoimmune, neoplastic and allergic diseases and conditions; psychopathological and neurological disease, graft versus host disease and allograft rejection.

The polynucleotides of these embodiments may be introduced to the subject's cells in vivo, or ex vivo, to isolated T-cells, as described above.

Downregulation of T-cell activity may be effected through modulation of intracellular signal transduction pathways, or reduction of cell surface structures responsible for Glutamate recognition, such as the Glutamate receptor family. Thus there is provided by the present invention a method of downregulating T-cell activity in a mammalian subject, the method effected by administering a therapeutically effective amount of a downregulating anti-Glutamine receptor antibody, the amount being sufficient to block Glutamate receptor activation, thereby downregulating T-cell activity in the subject. The downregulating anti-Glutamate receptor antibody may be monoclonal, or polyclonal, as detailed hereinabove. Likewise, the downregulating anti-Glutamate receptor antibody may be administered in vivo or ex vivo, as described hereinabove.

Patients having hyperproliferative disorders, which include both benign tumors and primary malignant tumors that have been detected early in the course of their development, may often be successfully treated by the surgical removal of the benign or primary tumor. If unchecked, however, cells from malignant tumors are spread throughout a patient's body through the processes of invasion and metastasis. Invasion refers to the ability of cancer cells to detach from a primary site of attachment and penetrate, e.g., an underlying basement membrane. Metastasis indicates a sequence of events wherein (1) a cancer cell detaches from its extracellular matrices, (2) the detached cancer cell migrates to another portion of the patient's body, often via the circulatory system, and (3) attaches to a distal and inappropriate extracellular matrix, thereby created a focus from which a secondary tumor can arise. Normal cells do not possess the ability to invade or metastasize and/or undergo apoptosis (programmed cell death) if such events occur (Ruoslahti, Sci. Amer., 1996, 275, 72).

Disseminating precancerous or cancerous cells often display ectopic expression of substrate binding molecules which may facilitate step (3) of the metastatic process as described above. Thus, modulation of the Glutamate receptor using the antisense compounds of the invention, and the decreased adhesion and migration of cancerous cells of T-cell origin may result in a decreased ability of the disseminating cancer cells to attach to a distal and/or inappropriate matrix, thereby modulating metastasis and invasion of non-cancerous tissues. The importance of substrate binding and migration to extravasation and metastatic spread of T-lymphoma and other cancer cells has been noted (see, for example, Wewer, U. M. et al., Proc Natl Acad Sci USA 1986; 83: 7137-41, and Hand, P. H. et al. Cancer Research 1985; 45: 2713-19).

While reducing the present invention to practice, it was noted that Glutamate stimulated binding to the major matrix protein fibronectin and chemotactic migration in human T-cells. Furthermore, Glutamate receptor (GluR3 type) surface expression was observed for the first time in cultured human T-cell leukemia (Jurkat) and mouse lymphoma (EL-4) cells. Thus, inhibition of sensitivity to Glutamate stimulation may be effective in downregulating binding and migration, providing a novel therapeutic approach for the treatment of primary T-cell cancer such as T-lymphoma and other myeloproliferative diseases.

While reducing the present invention to practice, it was observed that Glutamate induces expression of inhibitor of serine proteases Bomapin in cultured human T-cell lukemia (Jurkat) cells as well as in normal human T-cells. Since Bomapin has been associated with inhibition of induced apoptosis, inhibition of Glutamate sensitivity according to the present invention may be further effective in suppressing primary T-cell cancer growth and proliferation.

Thus, according to a further aspect of the present invention there is provided a method of preventing or treating a cancerous disease or condition in a mammalian subject, the method comprising introducing into the cell a polynucleotide which specifically inhibits Glutamate receptor production, the polynucleotide capable of reducing sensitivity to Glutamate stimulation, thereby reducing cancer cell proliferation and/or metastasis the subject. In preferred embodiments of the present invention the down-regulating polynucleotides are antisense, ribozyme and/or expressible polynucleotides encoding antisense or ribozyme oligoneucleotides capable of effectively reducing Glutamate receptor transcripts, as described above. Treatment of such cancerous disease or conditions may be in combination with one or more additional anticancer compounds and/or chemotherapeutic drugs. The downregulating polynucleotides of the invention are evaluated for their ability to modulate proliferation and metastasis using one or more assays known in the art and/or one or more appropriate animal models (for example, Thymidine uptake proliferation assay, extravasation assay, in-vivo T-cell homing assay).

In a preferred embodiment of the present invention, the expressible polynucleotide of the method is introduced into the cancerous cells in vivo. In another, more preferred embodiment, the expressible polynucleotide is introduced ex vivo, as described hereinabove.

Similarly, according to further aspects of the present invention there are provided methods for relieving or preventing undesired T-cell migration and function including T-cell mediated graft versus host disease, allograft rejection, neuronal damage, psychopathology, infectious, autoimmune and/or allergic diseases and conditions in a mammalian subject, the methods comprising introducing into at least one tissue of the subject a polynucleotide which specifically inhibits Glutamate receptor production, the polynucleotide capable of effectively reducing sensitivity to Glutamate stimulation of immune reactivity (e.g. T-cell activation, migration, extravasation and cytokine production), thereby reducing levels of infection and alleviating autoimmune and allergic conditions in the subject. In preferred embodiments of the present invention the downregulating polynucleotides are antisense, ribozyme and/or expressible polynucleotides encoding antisense or ribozyme oligoneucleotides capable of effectively reducing Glutamate receptor transcripts, as described above.

According to further aspects of the present invention there are provided additional methods of relieving or preventing the abovementioned autoimmune, neoplastic, hyperreactive, psychopathological, neurological and/or allergic conditions and diseases in a mammalian subject, the methods effected by administering to the subject a therapeutically effective amount of a downregulating anti-Glutamate receptor antibody, the amount being sufficient to effectively reduce Glutamate stimulation of immune reactivity, thereby alleviating the abovementioned T-cell related disease or condition in the subject. In one preferred embodiment, the downregulating anti-Glutamate receptor antibody may be monoclonal or polyclonal, prepared and characterized as described above. As detailed hereinabove, the down-regulating anti-Glutamate receptor antibody may be administered in vivo or ex vivo.

Likewise, autoimmune, hyperreactive, neoplastic, hyperreactive, psychopathological, neurological and/or allergic conditions and diseases in a mammalian subject may be treated or prevented by a down-regulating Glutamate analog. In one preferred embodiment, the down-regulating Glutamate antagonist analog is selected from a group including naturally occurring or synthetic analogs.

According to the present invention, there is provided a pharmaceutical composition comprising as an active ingredient at least one down-regulating Glutamate analog, being packaged and indicated for use in the prevention and/or treatment of a T-cell related condition, in which inhibiting T-cell activity is an effective therapy.

Further according to the present invention, there is provided a pharmaceutical composition comprising as an active ingredient a downregulating anti-Glutamate receptor antibody, being packaged and indicated for use in the treatment of a T-cell related condition in which inhibiting T-cell activity is an effective therapy.

The compositions of the present invention include bioequivalent compounds, including pharmaceutically acceptable salts and prodrugs. This is intended to encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to pharmaceutically acceptable salts of the nucleic acids of the invention and prodrugs of such nucleic acids. “Pharmaceutically acceptable salts” are physiologically and pharmaceutically acceptable salts of the nucleic acids of the invention: i.e., salts that retain the desired biological activity of the parent compound and do not impart undesired toxicological effects thereto (see, for example, Berge et al., “Pharmaceutical Salts,” J. of Pharma Sci. 1977, 66, 1-19). For therapeutic or prophylactic treatment, amino acids, amino acid derivatives, polynucleotides and antibodies are administered in accordance with this invention. Components of the invention may be formulated in a pharmaceutical composition, which may include pharmaceutically acceptable carriers, thickeners, diluents, buffers, preservatives, surface active agents, neutral or cationic lipids, lipid complexes, liposomes, penetration enhancers, carrier compounds and other pharmaceutically acceptable carriers or excipients and the like in addition to the amino acids, amino acid derivatives, polynucleotides and antibodies. Such compositions and formulations are comprehended by the present invention.

As used herein, the term “pharmaceutically acceptable carrier” (excipient) indicates a pharmaceutically acceptable solvent, suspending agent or any other pharmacologically inert vehicle for delivering one or more nucleic acids to an animal. The pharmaceutically acceptable carrier may be liquid or solid and is selected with the planned manner of administration in mind so as to provide for the desired bulk, consistency, etc., when combined with a nucleic acid and the other components of a given pharmaceutical composition. Typical pharmaceutically acceptable carriers include, but are not limited to, binding agents (e.g., pregelatinized maize starch, polyvinyl-pyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrates (e.g., starch, sodium starch glycolate, etc.); or wetting agents (e.g., sodium lauryl sulphate, etc.). Sustained release oral delivery systems and/or enteric coatings for orally administered dosage forms are described in U.S. Pat. Nos. 4,704,295; 4,556,552; 4,309,406; and 4,309,404.

The compositions of the present invention may additionally contain other adjunct components conventionally found in pharmaceutical compositions, at their art-established usage levels. Thus, for example, the compositions may contain additional compatible pharmaceutically-active materials such as, e.g., antipruritics, astringents, local anesthetics or anti-inflammatory agents, or may contain additional materials useful in physically formulating various dosage forms of the composition of present invention, such as dyes, flavoring agents, preservatives, antioxidants, opacifiers, thickening agents and stabilizers. However, such materials, when added, should not unduly interfere with the biological activities of the components of the compositions of the invention.

Regardless of the method by which the amino acids, amino acid derivatives, polynucleotides and antibodies of the invention are introduced into a patient, colloidal dispersion systems may be used as delivery vehicles to enhance the in vivo stability of the and/or to target the amino acids, amino acid derivatives, polynucleotides and antibodies to a particular organ, tissue or cell type. Colloidal dispersion systems include, but are not limited to, macromolecule complexes, nanocapsules, microspheres, beads and lipid-based systems including oil-in-water emulsions, micelles, mixed micelles, liposomes and lipid:peptide, polynucleotide and/or antibody complexes of uncharacterized structure. A preferred colloidal dispersion system is a plurality of liposomes. Liposomes are microscopic spheres having an aqueous core surrounded by one or more outer layers made up of lipids arranged in a bilayer configuration (see, generally, Chonn et al., Current Op. Biotech. 1995, 6, 698-708).

For therapeutic uses, the pharmaceutical compositions of the present invention may be administered in a number of ways depending upon whether local or systemic treatment is desired and upon the area to be treated. Administration may be topical (including ophthalmic and to mucous membranes including vaginal and rectal delivery) pulmonary, e.g., by inhalation or insufflation of powders or aerosols, including by nebulizer, intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

For certain conditions, particularly skin conditions including but not limited to, psoriasis, administration of compounds to the skin is preferred. Administration of compounds to the skin may be done in several ways including topically and transdermally. A preferred method for the delivery of biologically active substances to the skin is topical administration. “Topical administration” refers to the contacting, directly or otherwise, to all or a portion of the skin of an animal. Compositions for topical administration may be a mixture of components or phases as are present in emulsions (including microemulsions and creams), and related formulations comprising two or more phases. Transdermal drug delivery is a valuable route for the administration of lipid soluble therapeutics. The dermis is more permeable than the epidermis and therefore absorption is much more rapid through abraded, burned or denuded skin. Inflammation and other physiologic conditions that increase blood flow to the skin also enhance transdermal adsorption. Absorption via this route may be enhanced by the use of an oily vehicle (inunction) or through the use of penetration enhancers. Hydration of the skin and the use of controlled release topical patches are also effective ways to deliver drugs via the transdermal route. This route provides an effective means to deliver drugs for both systemic and local therapy.

In addition, iontophoresis (transfer of ionic solutes through biological membranes under the influence of an electric field) (Lee et al., Critical Reviews in Therapeutic Drug Carrier Systems, 1991, p. 163), phonophoresis or sonophoresis (use of ultrasound to enhance the absorption of various therapeutic agents across biological membranes, notably the skin and the cornea) (Lee et al., Critical Reviews in Therapeutic Drug Carrier Systems, 1991, p. 166), and optimization of vehicle characteristics relative to dose:deposition and retention at the site of administration (Lee et al., Critical Reviews in Therapeutic Drug Carrier Systems, 1991, p. 168) may be useful methods for enhancing the transport of drugs across mucosal sites in accordance with the present invention.

In addition, according to the present invention there is provided an assay of determining an effect of Glutamate or a Glutamate analog on a T-cell related disease or condition, the assay effected by exposing an organism having the aforementioned T-cell related disease or condition to Glutamate or a Glutamate analog and assessing at least one T-cell related symptom of the disease in that organism.

Further, according to the present invention there is provided an assay of determining the sensitivity of a T-cell to Glutamate or a Glutamate analog, the assay effected by exposing the T-cell to one or more concentrations of Glutamate or a Glutamate analog and assessing the stimulatory state of the T-cell. In a preferred embodiment the Glutamate or Glutamate analog concentration may be 0.1 ng/ml to 1 mg/ml, sufficient to produce a significant alteration in T-cell function, as measured by, for example, radiolabeled precursor uptake, mitotic index, specific gene expression, adhesion, migration and the like (see Examples section that follows). The assay may be performed in vitro or in vivo. The assay of the present invention may be used to determine the sensitivity of a T-cell to a downregulating Glutamate analog. By varying the assay conditions, the sensitivity of a T-cell to Glutamate analog inhibition of T-cell activity may be assessed. The Glutamate analog may a naturally occurring or synthetic analog.

Similarly, the assay of the present invention may be applied to additional methods of downregulating T-cell activity. Thus, the sensitivity of a T-cell to downregulating Glutamate analogs, or to polynucleotides downregulating Glutamate receptor expression and/or to downregulating anti-Glutamate receptor antibodies may be assayed. Exposure of the T-cells to the downregulating modulators may be performed in vivo or in vitro, as described in the Examples section that follows. The expressible polynucleotides may be capable of transient or stable expression in the T-cell. Likewise, the effect of the abovementioned methods of downregulating may be assayed in an organism suffering from an autoimmune, infectious, allergic, neoplastic, psychopathological or other disease or condition requiring reduced T-cell activity (see abovementioned list of conditions).

Consistent with, and in addition to the methods for modulation of Glutamate stimulation of T-cell activity detailed herein, endogenous production of Glutamate in the CNS may be increased or inhibited by physiological or non-physiological factors. In addition, Glutamate receptor expression, and cytokine secretion by T-cells may be modulated. Such modulation of endogenous cytokine secretion and Glutamate receptor expression can further regulate Glutamate-associated activity in T- and other Glutamate-sensitive cells.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes 1-111 Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Experimental Methods

Human T-cells:

Human T-cell clones were prepared essentially as described by Besser M and Wank J (Besser M and Wank J, J Immunol 1999; 162:6303-306): Human PBMC were purified from whole blood by Ficoll gradient (Amersham Pharmacia Biotech, Freiburg, Germany). CD4⁺ Th (T-helper) cells were isolated by using CD4 mAbs coupled to magnetic beads (Dynal, Hamburg, Germany). T cells were cultured at 1-2×10⁶ cells/ml either in the presence of 1% PHA (Difco, Hamburg, Germany), 20 μg of anti-CD3 (OKT-3, Cilag, Sulzbach, Germany)-precoated plates in combination with IL-2 (20 U/ml) or IL-4 (100 U/ml), or with 100 ng/ml neurotrophins (Prepro, London, England), 20 U/ml IL-2 (Chiron, Ratingen, Germany), 100 U/ml IL-4 (Genzyme, Ruesselsheim, Germany) or 500 U/ml IFN-γ (Thomae, Bicherach an der Riss, Germany) alone. RPMI 1640 culture medium (Life Technologies, Eggenstein, Germany) was supplemented with 10% FCS (Life Technologies). Alloprimed PBMC were seeded at 0.4 cell/well on allogeneic specific feeder layer. Clonality was confirmed by FACS analysis; T cell clones 234 and 305 were also recloned at a cell concentration of 0.1 cell/well. Constitutive expression in immune cell bulk cultures was assessed immediately after cell separation, in cloned T lymphocytes after 3 days without further addition of IL-2.

Fresh, normal human T-cells were purified from the peripheral blood of healthy donors as follows: blood was diluted 1:1 in sterile phosphate-buffered saline (PBS) and the leukocytes were isolated on a Ficoll gradient. After washing, the cells were incubated on nylon-wool columns (Novamed Ltd., Jerusalem, Israel). One hour later, non-adherent T-cells were eluted, washed, and counted.

The T-cells were suspended at a concentration of 1.5×10⁶ cells per ml in RPMI Medium (Sigma, St. Louis, Mo.), containing 10% FCS, Penicillin/Strepomycin/Ampicillin and L-Glutamine (Biological Industries, Beit HaEmek, Israel).

Neurotransmitters, agonists and antagonists were added and the cells were incubated at 37 C°, 5% CO₂ for up to 72 hours, as indicated for individual experiments. The incubated T-cells were then collected and used for further experimentation.

Neurotransmitters, Agonists and Antagonists:

The following neurotransmitters and functional analogs were used throughout this study: Glutamate (Sigma, St. Louis, Mo.) and the Glutamate antagonist CNQX (Tocris, UK).

Glutamate was added to fresh T-cells at a final concentration of 10⁻⁸ M. CNQX was used at a final concentration of 10⁻⁶M.

Antibodies:

The following antibodies were used: anti-human CD3 mAb, anti-human CD28 mAb (Pharmingen, BD, San Jose Calif.), rat polyclonal anti-GluR3 antibody from rats immunized in our laboratory with a specific peptide derived from the extracellular domain of the GluR3 (termed GluR3B, a.a. 372-395) (Levite M et al J Autoimmunity 1999; 13:61-72) and FITC-conjugated goat anti-rat IgG (Jackson Labs, Bar Harbor, Me., USA).

T-Cell Adhesion Assay:

Adhesion of T-cells to fibronectin and laminin was assayed as follows: normal human T-cells, purified from a fresh blood sample, were suspended (1×10⁶ cells/ml) in rest medium (RPMI-1640, supplemented with 10% fetal calf serum (Sigma Chemical Co., St. Louis, Mo.), 1% antibiotics, 1% glutamine (Biological Industries, Beit Haemek, Israel) and 0.4% fungizone (GibcoBRL, Life Technologies Ltd., Paisley, Scotland)). The cells were then supplemented with 10 nM Glutamate, or Glutamate receptor agonists/antagonists, and incubated for variable periods of time (0.5-72 hours, 37° C., 7.5% CO₂ humidified incubator). Following incubation the cells were washed and resuspended in adhesion medium (RPMI-1640 supplemented with 0.1% bovine serum albumin (BSA, Sigma)). The cells were then seeded in 96 well flat-bottomed microtiter plates (Falcon, Becton Dickinson, Heidelberg, Germany, 1×10⁵ cells/100 μl/well pre-coated with fibronectin or laminin (ICN Biomedicals Inc., Aurora, Ohio, 0.5 mg/well, 18 hours, 4° C.). Cells treated with phorbol 12-myristate 13-acetate (PMA, Sigma, 10 ng/ml) served as a positive control. The adhesion plates were incubated (37° C., 30 minutes, 7.5% CO₂ humidified incubator), and then washed several times with PBS to remove non-adherent T-cells. The adhered cells were lysed by adding 60 μl/well of lysis-substrate solution (0.5% Triton X-100 in water mixed with an equal volume of 7.5 mM p-nitrophenol-N-acetyl-β-D-glucosaminide (Sigma, St. Louis, Mo.) in 0.1M citrate buffer pH=5.0). The plates were then incubated for 18 h in a CO₂-devoid 37° C. incubator, and the reaction was stopped by the addition of 90 ml/well of 50 mM glycine (Sigma, St Louis, Mo.) pH=10.4, containing 5 mM EDTA. The optical density (OD) was measured at 405 nm in a standard ELISA reader. The OD was converted to actual number of cells using a standard curve performed in each experiment.

In-Vitro Migration Assay:

Normal human T-cells (1×10⁶ cells/well in rest medium) were pretreated with Glutamate (10 nM, >18 hours, 37° C., 7.5% CO₂ humidified incubator), washed, resuspended in adhesion medium and fluorescent labeled (50 μg/ml, 30 minutes, 37° C., 7.5% CO₂ humidified incubator) with 2′,7′-bis-(2-carboxyethyl)-5-(and-6)-carboxyfluorescein acetoxymethyl (BCECF AM, Molecular Probes, Eugene, Oreg.). The cells were then washed, resuspended in adhesion medium, and added to the upper chambers (2×10⁵ cells per 100 μl well) of a 24-well chemotaxis microchamber plate (Corning Inc., Corning, N.Y.). The two compartments of the microchambers were separated by polycarbonate filters (5.0 mm pore size) pre-coated with fibronectin or laminin (25 mg/ml, about 1.5 hour, 37° C.). The lower chambers contained adhesion medium, which was supplemented, where indicated, with 100-250 ng/ml of the chemokine stromal cell-derived factor 1a (SDF-1a, Peprotech Inc., Rocky Hill, N.J.). The chemotaxis microchamber plate was incubated (3 hours, 37° C., 7.5% CO₂ humidified incubator), the filter-containing upper chambers were gently removed and the contents of the individual lower chambers (containing the migrated cells) thoroughly mixed by pipetting and transferred into clean tubes. The number of cells in each tube was determined by FACSORT. Counting time for all the experimental groups was two minutes.

T-Cell Receptor (TCR) Activation:

Normal human T-cells, separated from blood samples of healthy donors, were activated via their TCR as follows: 24-well plates (non-tissue culture treated, Becton-Dickinson, N.J.) were precoated with 0.5 ml/well of PBS containing a mixture of anti-human CD3 (Pharmingen, BD, San Jose, Calif.) and anti-human CD28 (Pharmingen, BD, San Jose, Calif.) mAbs (1 μg/ml final of each Ab, 4° C., overnight). The plate was then washed with PBS, blocked (0.5 ml/well of PBS containing 1% BSA (Sigma), 20-30 minutes, 37° C., 7.5% CO₂ humidified incubator), and washed again with PBS. After washing, the cells were seeded into the mAbs-coated wells (1-1.5×10⁶ cells/ml “rest medium”/well) and incubated (24-72 hours, 37° C., 7.5% CO₂ humidified incubator). After incubation, the activated cells were collected, examined microscopically, counted and used for further experiments.

Analysis of Gene Expression Using the Human Atlas cDNA Expression Array:

Poly A+ RNA was extracted from human T-lymphocytes before and after treatment with 10 nM Glutamate for 72 hours, using the Atlas Pure Total RNA Labeling System (Clontech Laboratories, Inc. Palo Alto, Calif.) according to manufacturers recommendations. Following DNase treatment, ³²P-labeled cDNA was prepared from poly A+ RNA preparations that were prepared from either untreated or Glutamate treated human T-cells. Hybridizations to the Atlas Human cDNA Expression Arrays membranes (Catalog No. 1.2 KIII (7850-1) and III (7855-1), Clontech Laboratories) were performed by Clonetech Laboratories, as described in the user manual. Analysis of the expression pattern of up and down regulated genes, as compared to that of untreated T-cells, was performed by Clontech, as described in their manual.

Reversed Transcription (RT) PCR:

Bomapin Expression

Total RNA was extracted by using Trizol RNA isolation reagent (Molecular Research Center, Cincinnati, Ohio) based on the acid guanidinium thiocyanate-phenol-chloroform extraction method, according to manufacturer recommendations. RT-PCR was used to amplify the levels of endogenous Bomapin mRNA that may be present in the peripheral human T-cells and in the Jurkat cells (a human mature leukemic cell line that phenotypically resembles resting human T lymphocytes). The expression of the ribosomal protein S-14, derived from the same tissue preparations, served as an internal control. Each reaction contained four oligonucleotides primers, two for Bomapin and two for the internal control S-14. PCR conditions were: cDNA equivalent to 50 ng RNA was amplified for 30 cycles, the annealing temperature was 60° C. and the final MgCl₂ concentration was 2.5 mM. The Taq DNA polymerase used in this study was the BIO-X-ACT DNA polymerase (Bioline UK Ltd., London. UK). The PCR products were separated electrophoretically on a 1.5% agarose gel containing ethidium bromide, and visualized under UV light.

GluR3 Expression

Total RNA was extracted from cultured T-cells using Trizol RNA isolation reagent (Molecular Research Center, Cincinnati, Ohio) based on the acid guanidinium thiocyanate-phenol-chloroform extraction method, according to manufacturer recommendations. RT-PCR was used to amplify the levels of endogenous GluR3 mRNA present in the cultured human T-cells following 30 minutes exposure to Glutamate. The expression of the ribosomal protein S-14, derived from the same cell preparations, served as an internal control. Each reaction contained four oligonucleotides primers, two for GluR3 and two for the internal control S-14. PCR conditions were: cDNA equivalent to 50 ng RNA was amplified for 30 cycles, the annealing temperature was 60° C. and the final MgCl₂ concentration was 2.5 mM. The Taq DNA polymerase used in this study was the BIO-X-ACT DNA polymerase (Bioline UK Ltd., London. UK). The PCR products were separated electrophoretically on a 1.5% agarose gel containing ethidium bromide, and visualized under UV light.

Oligonucleotide Primers:

For the PCR reactions the following specific Bomapin, S-14 and GluR3 oligonucleotide primers were used:

Bomapin—5′ GCAGTGGGCCTTCAACTCTAC 3′ (SEQ ID NO: 4) and 5′ GGATGGGACTCTAATTCGTATATC 3′ (SEQ ID NO: 5) corresponding to nucleotides 706-726(sense) and 1078-1101-(antisense) respectively. The predicted size of band is 396 base pairs.

S-14—5′CAGGTCCAGGGGTCTTGGTCC 3′ (SEQ ID NO: 6) and 5′ GGCAGACCGAGATGAATCCTCA 3′ (SEQ ID NO: 7) corresponding to nucleotides 180-203 (sense) and 322-345 (antisense) respectively. The predicted size of the band is 166 base pairs.

GluR3—5′CGATACTTGATTGACTGCGA 3′ (SEQ. ID NO: 8) and 5′ TACTATGGTCCGATTCTCTG 3′ (SEQ ID NO: 9) corresponding to nucleotides 699-718 (sense) and 1312-1331 (antisense) respectively. The predicted size of the band is 663 base pairs.

DNA Sequencing:

The appropriate cDNA fragments of Bomapin from the peripheral human T cells were extracted from the gels by using the QIAquick Gel Extraction Kit (QIAGEN GmbH, Hilden, Germany). The nucleotide sequencing of the specifPCR bands were obtained by automated direct DNA sequencing, according to the manufacturers recommendations (PE Applied Biosystems; model 377, Perkin Elmer Corp, Foster City, Calif.).

Immunofluorescence Staining for the Ionotropic Glutamate Receptor Subtype3 (GluR3):

Normal human T-cells, isolated from fresh peripheral blood lymphocytes, or antigen-specific mouse T-cells (directed against myelin basic protein 87-99)(Levite M et al Proc Natl Acad Sci USA 1998; 95:12544-49) were subjected to double immunofluorescence staining, using a rat polyclonal anti-human GluR3 antibody, (100 μl of 10-50 μg/ml dilution per 1×10⁶ cells/tube; 30 minutes on ice), or normal rat sera for control. The rat polyclonal antibody was purified in our laboratory, from rats immunized with a specific peptide derived from the extracellular domain of the GluR3 (termed GluR3B, a.a. 372-395). The cells were then stained with an FITC-conjugated goat anti-rat IgG (50 μl of 1:100 dilution). The surface expression of the GluR3 on the human T-cells was also confirmed using a commercially available polyclonal goat anti-human/rat/mouse antibody (100 μl of 1:500 dilution per 1×10⁶ cells/tube; 30 minutes on ice) (Dianova, Hamburg, Germany). Cells staining with normal rat serum, or staining only with the second and third antibodies served as additional negative controls. Fluorescence profiles were recorded in a FACSORT.

Cytokine Determination by ELISA:

Cytokine levels were measured in supernatants from Glutamate stimulated human T-cell cultured as described hereinabove. Isolated human T-cells (1-5×10⁴) were incubated in round-bottom 96 well plates (Nunc, VWR Scientific Products, Westchester Pa.) with 10⁻⁸ M Glutamate for 24-72 hours, and cytokine levels measured by quantitative sandwich ELISA, using pairs of antibodies obtained from Pharmingen (BD Pharmingen, San Diego Calif.), according to the manufacturers instructions. Where indicated, Glutamate stimulation of cytokine secretion was compared to activation via T-cell receptor, as described hereinabove. Results are expressed in pg/ml as mean ISD concentration of duplicate culture supernatants.

Statistical Analysis:

Statistical significance was analyzed by Student's t test.

### Experimental Results

### Example 1

T-Cells Respond to Direct Stimulation With Glutamate by Initiation, Modulation or Suppression of de Novo Gene Expression

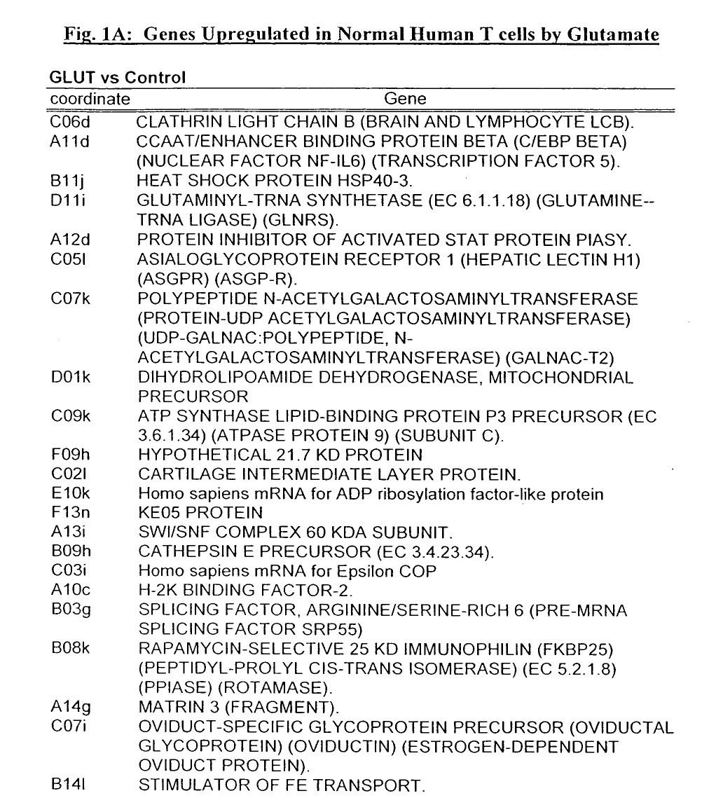

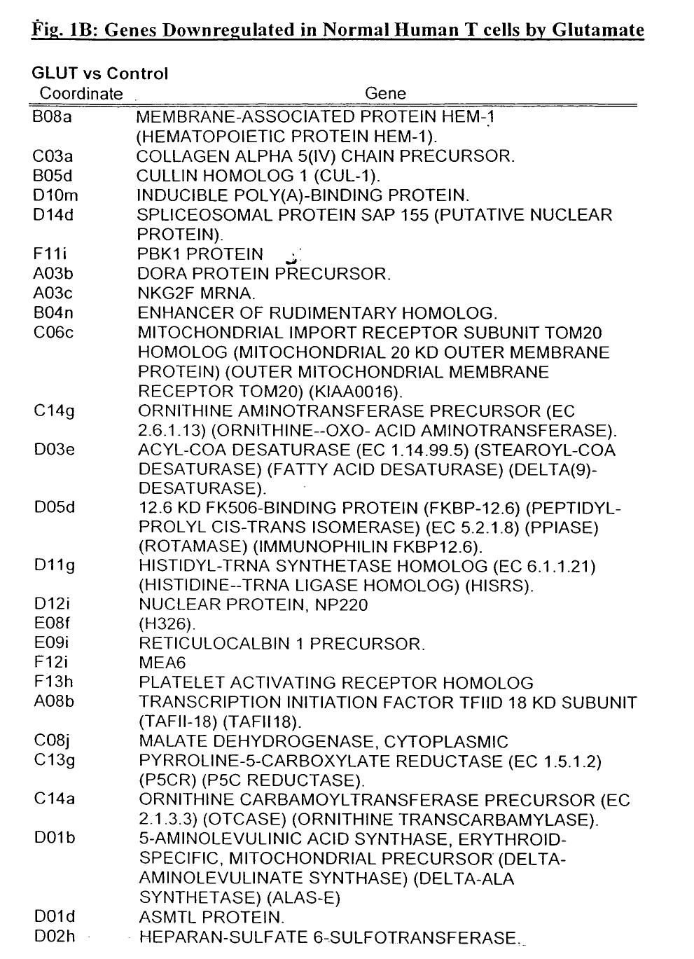

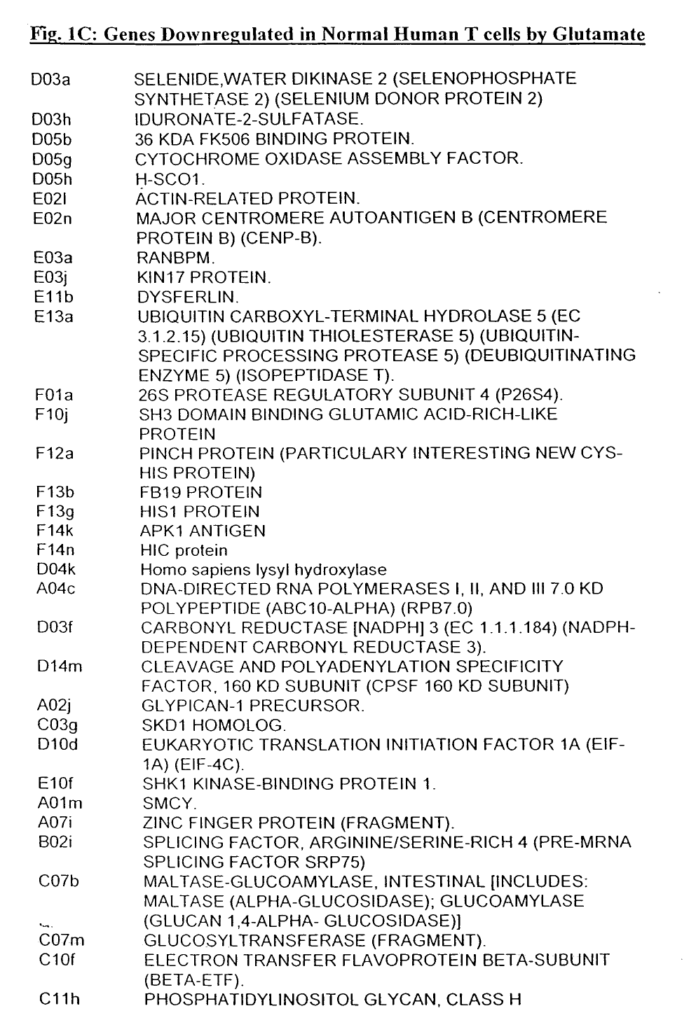

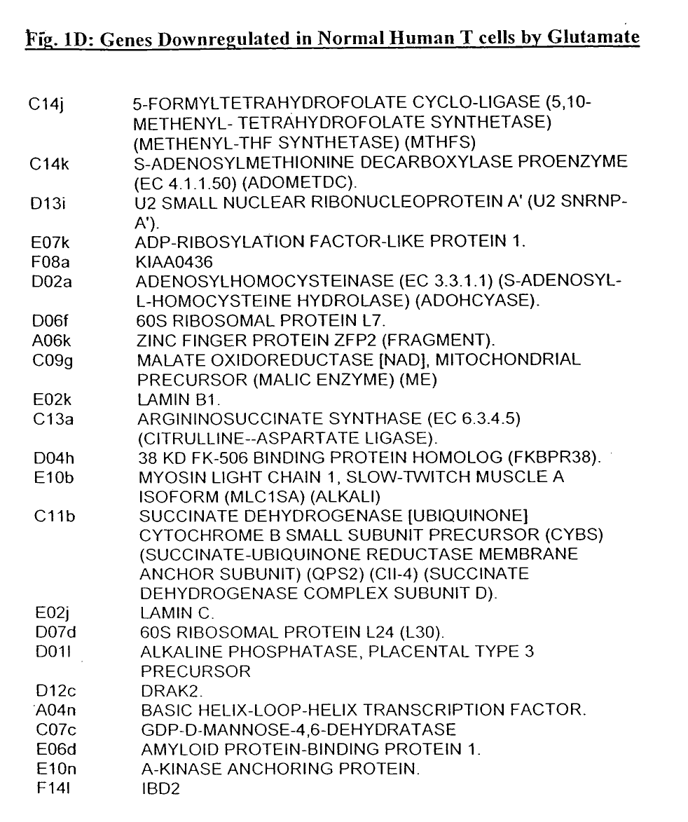

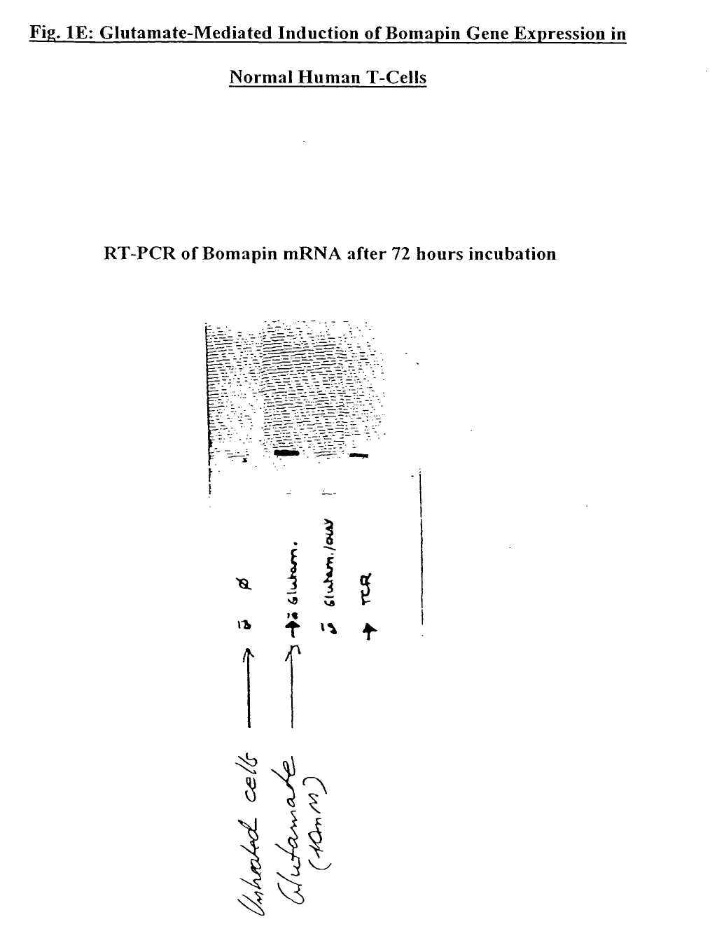

To explore the possible direct effects of Glutamate on gene expression by T-cells, resting human peripheral T-cells were exposed to Glutamate (10 nM) for 72 hours. Poly A+ RNA was prepared from both Glutamate-treated and untreated cells and reverse transcribed to ³²P-labeled cDNA. Using an Atlas human cDNA expression array (i.e. a positively charged nylon membrane spotted with 1200 different human cDNAs) for identification of effected genes, the reverse transcribed products were characterized by hybridization to the atlas membranes. The differential pattern of expression between untreated cells and Glutamate-treated cells was visualized by autoradiography, and quantified by densitometry [see FIGS. 1A and 1B-D for a continuous list of examples of up- and downregulated genes]. The results revealed that Glutamate induced the over expression of mRNA encoding for several genes (FIG. 1A), and down-regulated the expression of others (FIGS. 1B-D). Surprisingly, in addition to modulating the expression of a number of typical T-cell genes (for example, Rapamycin-selective 25 KD Immunophilin; Heat Shock protein 40; and Cathepsin E precursor), exposure to Glutamate triggered the expression of a number of genes previously undetected in T-cells. Thus, as noted in FIG. 1A, the neurotransmitter glutamate induced expression of Stimulator of Fe Transport (SFT), oviductal glycoprotein, Clathrin light chain B (LCB), Glutaminyl t-RNA synthase, Protein Inhibitor of Activated STAT (PIAS), Cartilage Intermediate Layer Protein (CILP) and Matrin 3, previously detected in non-lymphoid tissue only. Furthermore, Clathrin LCB and SFT have been directly implicated in the pathogenesis of Alzheimers disease and anemia of chronic disorders, respectively, suggesting a role for Glutamate in the regulation of immune function in these conditions.

One example of the Glutamate's modulation of pathology-related T-cell gene expression is the induction of expression of the serine protease inhibitor Bomapin (protease inhibitor 10, PI 10, not shown in FIG. 1A). This member of the ovalbumin family of serine protease inhibitors is expressed at elevated levels in patients with acute myeloid leukemia and chronic myelomonocytic leukemia, inhibits TNF alpha-induced cell death, and has been linked to the regulation of protease activities in early hematopoiesis (Riewald, M et al Blood 1998; 91:1256-62 and Schleef R R and Chuang T L J Biol Chem 2000; 275:26385-9). RT-PCR analysis of the mRNA of peripheral T-cells incubated with and without 10 nM Glutamate clearly demonstrates the increased abundance of Bomapin transcripts following Glutamate treatment (FIG. 1E, Glutam., arrow). Bomapin expression is also induced by T-cell receptor-mediated activation of T-cells (FIG. 1E, TCR). The specificity of Glutamate induction of Bomapin expression is further demonstrated by the absence of detectable Bomapin transcripts in T-cells treated with Glutamate in the presence of the Glutamate GluR3 receptor-antagonist CNQX (FIG. 1E, Glutam./CNQX).

Taken together, these results constitute the first demonstration of the direct action of Glutamate on T-cell activation, resulting in a Glutamate-specific pattern of de novo gene transcription.

### Example 2

Glutamate Induces Cytokine Secretion in Resting Human T-Cells.

T-cell activation is characterized by numerous responses, such as proliferation, adhesion, chemotaxis and cytokine secretion. It is via the release of specific factors such as the cytokines, that the cells of the immune system communicate with each other to coordinate appropriate immune and inflammatory responses. Typically, cytokine secretion in unstimulated T-cells is minimal, but can be strongly induced by activation with well-known inducers such as a specific antigens, mitogens, cytokines, and TCR activating antibodies. T-helper cell subpopulations Th0, Th1 and Th2 cells are characterized by the types of cytokines which they synthesize and secrete: Pluripotent, non-committed Th0 cells secrete a variety of cytokines, committed Th1 typically secrete IL-2 and IFN-γ, and Th2 secrete IL-4, IL-5, and IL-10, IL-13 and other Th2 cytokines. Many normal and pathological conditions are associated with specific cytokine profiles, and as a rule, Th1 cells induce disease while the Th2 cells are active in their prevention.

Due to the primary importance of T-cell cytokines in disease and health, the ability of Glutamate to induce cytokine secretion in normal and cloned human T-cells was investigated.

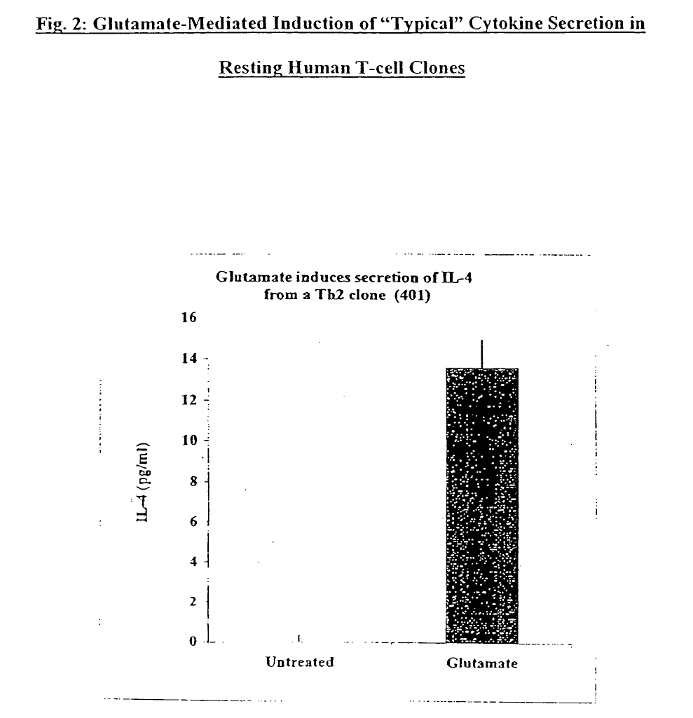

FIG. 2 shows the response of cloned human Th2 cells when incubated 24-72 hours in the absence or presence of 10⁻⁸ M Glutamate without antigen stimulation. Whereas none of the typically Th2 specific cytokine IL-4 was detected by ELISA in the untreated cells (Untreated), incubation with Glutamate induced a strong release of IL-4 (Glutamate).

Although a rare occurrence, induction of atypical, or “forbidden” cytokine secretion has been observed in vitro, resulting in the “reversion” of a T-cell response, in the presence of antigens and antigen-presenting cells (Mocci S and Coffman R L J Immunol 1997; 158:1559-64), and also by neuropeptides (Levite M et al PNAS USA 1998; 95:12544-54), suggesting that non-T-cell receptor stimulation participates in determining the specificity of immune and inflammatory responses. However, Glutamate modulation of cytokine secretion profile has never been demonstrated. Thus, the effect of Glutamate on the cytokine profile of resting (no antigen stimulation) human T-cell clones was measured by ELISA using cytokine-specific antibodies.

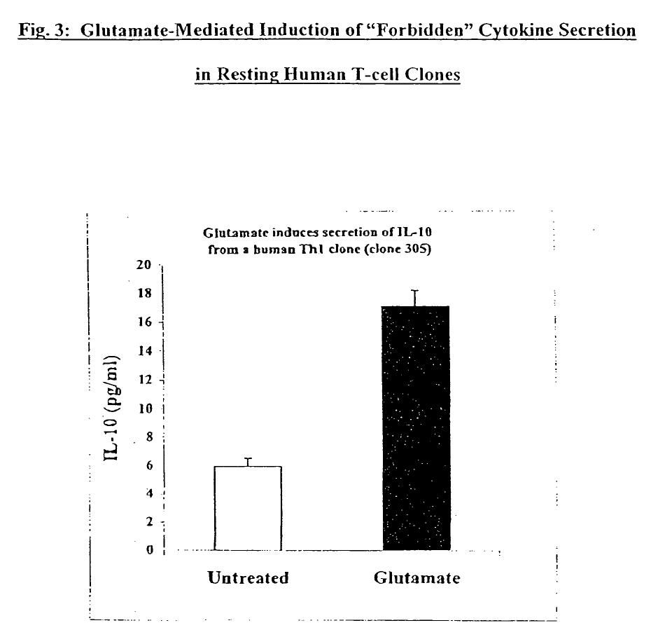

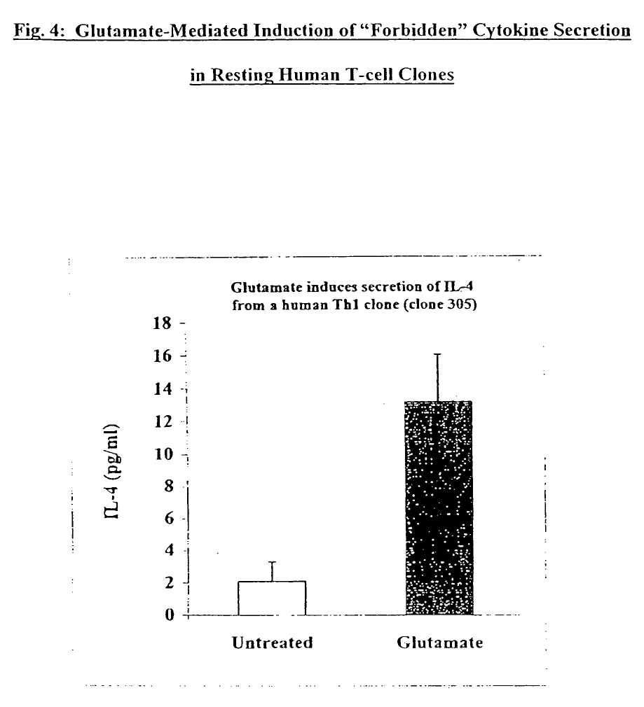

Incubation of cloned, resting human Th1 cells (clone 305) with 10⁻⁸ M Glutamate caused a significant induction of the “atypical” cytokines IL-10 (FIG. 3) and IL-4 (FIG. 4) secretion (Glutamate), as compared with untreated cultures (Untreated). IL-4 and IL-10 are typically secreted by Th2 cells, and are considered “forbidden” for Th1 cells (Martino G et al Ann Neur 1998; 43:340-49). Thus, Glutamate alone, in the absence of additional stimulators, directly activates human T-cell cytokine secretion, and is similarly capable of directly modulating the cytokine profile of committed T-cell clones.

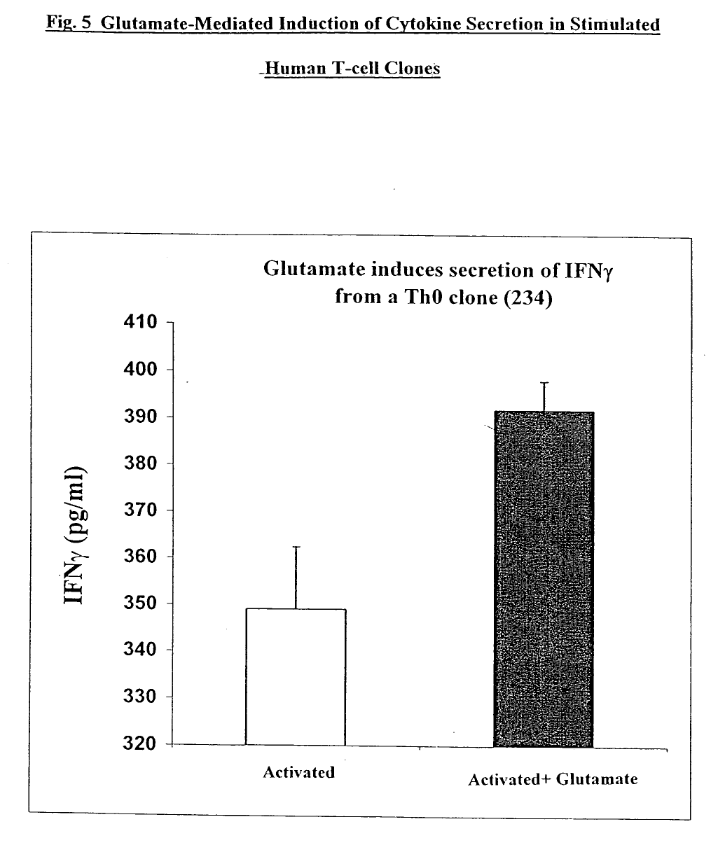

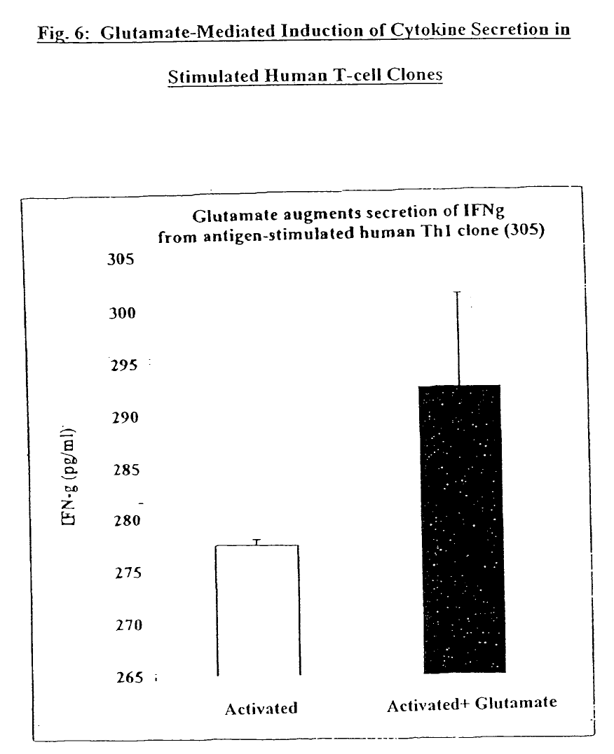

To gain further insight into the independent and additive nature of Glutamate- and antigen-mediated effects on cytokine secretion in human T-cells, IFN-γ (a cytokine typically secreted by both Th0 and Th1 cells) levels were measured in cultures of cloned human Th0 and Th1 cells stimulated with both specific antigens and Glutamate. FIGS. 5 and 6 demonstrate the effects of 20 hours incubation with 10⁻⁸M Glutamate and fully mismatched allopriming B cells on the secretion of IFN-γ from cloned Th0 (234) and Th1 (305) cells, respectively, compared to antigen stimulation alone (Untreated). Clearly, Glutamate efficiently stimulates additional significant IFN-γ secretion from both activated T-cell clones.

Taken together, these results clearly demonstrate the ability of Glutamate to activate T-cell function, in this case cytokine secretion, to influence T-cell function and destiny, as in reversal of typical cytokine profiles, and to act independently of, and in addition to other effectors of T-cell activation, such as mismatched HLA molecules. Thus, Glutamate may play a role in both the initial phases of general and, specifically neuroimmune response, and in the modulation of immunity and inflammation in the central nervous system and wherever else T-cells are found in the course of disease and/or pathology.

### Example 3

Glutamate Induces T-Cell Adhesion to Extra Cellular Matrix Proteins Via Specific Ionotropic Glutamate Receptor.

To study the functional consequences of Glutamate mediated T-cell activation, the ability of Glutamate-treated normal human T-cells to adhere to laminin and fibronectin was assessed. It is widely accepted that only activated T-cells can bind to components of the basement membrane and extracellular matrix, such as laminin and fibronectin. In order to determine Glutamate's ability to induce such cell binding, the adhesion to laminin- or fibronectin-coated microtiter plates of Glutamate-treated cells was compared to that of untreated cells (negative control, BG).

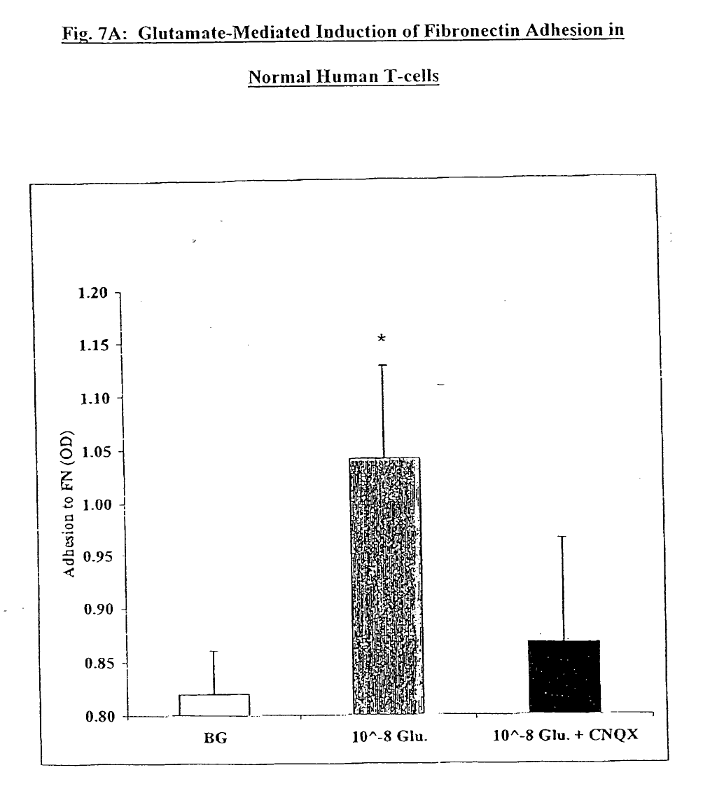

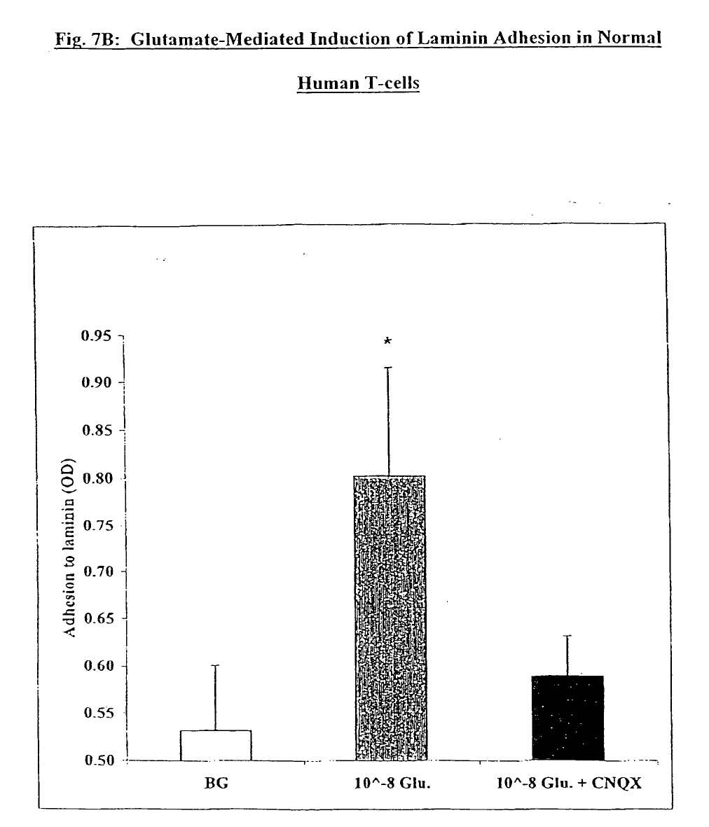

FIG. 7A demonstrates the relative proportions of treated and untreated fresh human T-cells adhering to fibronectin, expressed in terms of OD₄₅₀. Incubation of the cells with physiological concentrations (10⁻⁸M) of Glutamate (30 minutes) clearly induces a significant increase in fibronectin and laminin binding.

Glutamate receptors are commonly divided into two major groups: metabotropic, and ionotropic, the latter effecting changes in ion permeability of membranes. The receptor subtype mediating Glutamate induction of adhesion to laminin and fibronectin was investigated by examining the effect of addition of CNQX (a specific AMPA ionotropic Glutamate receptor antagonist) to Glutamate during incubation of the T-cells. FIGS. 7A and 7B show that CNQX strongly inhibits Glutamate-mediated fibronectin and laminin binding (Glu+CNQX). The results clearly indicate that Glutamate markedly induces adherance of normal human T-cells to extracellular matrix proteins, mediated by stimulation of previously uncharacterized specific ionotropic lymphocyte Glutamate receptors.

### Example 4

Glutamate Augments the in Vitro Chemotactic Migration of T-Cells.

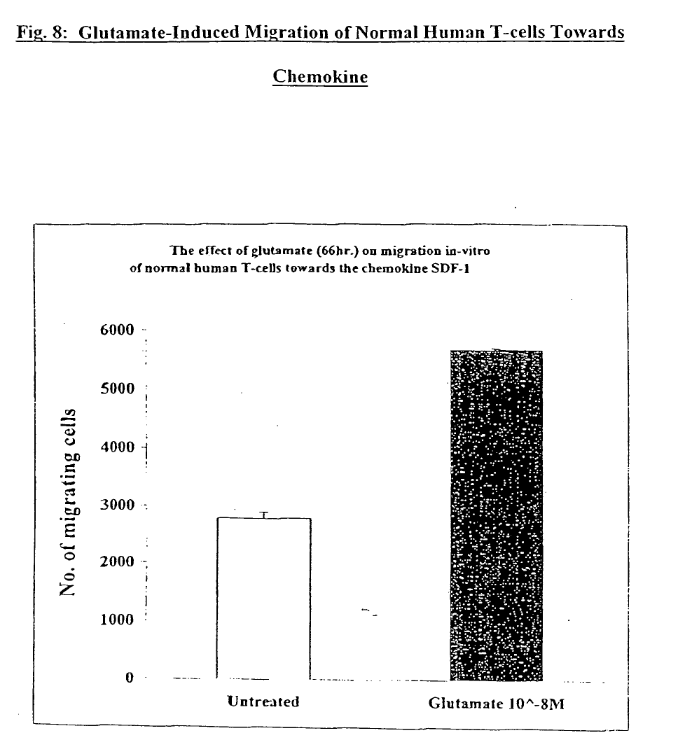

Adhesion of T-cells to components of the basement membrane is a crucial step in the series of events that eventually enable T-cells to migrate and extravasate from the blood stream to specific tissues. T-cells, which constantly move randomly, exhibit the crucial ability to move in a directional manner by responding to remotely secreted chemoattractants, via specific surface-expressed chemokine receptors. To determine whether Glutamate can induce T-cells to migrate towards a chemoattractant, we made use of the chemotaxis microchamber migration assay and scored the number of fluorescence-labeled normal human T-cells migrating from a medium-containing upper chamber to a chemoattractant-containing lower chamber. The chambers were separated by filters pre-coated with laminin or fibronectin, thus making the adhesion to the extra cellular matrix proteins a necessary (but not sufficient) step for the migration to the lower chamber. The potent stromal cell-derived factor-1 (SDF-1) chemokine, which has a specific receptor on the T-cell surface termed CXCR4, was used as a chemoattractant source. The number of migrating T-cells to chemokine-devoid lower chambers constituted background (BG) migration. The results of one representative experiment with fibronectin (FIG. 8), expressed as the number of migrating cells, indicate that pre-treatment of normal human T-cells for 66 hours with even low concentrations of Glutamate (Glutamate 10⁻⁸) significantly augments their migration towards the chemoattractant SDF-1. Further experimentation indicated optimum binding occurs with shorter (18-24) incubation times.

### Example 5

Human T-Cells Express the Ionotropic GluR3 Receptor and Downregulate its Level in Response to Glutamate Stimulation.

No previous studies have demonstrated the expression of specific ionotropic Glutamate receptors in isolated human T-cells or T-cell clones. To study the possible relevance of Glutamate-T cells interactions to autoregulation and fine tuning of neuroimmune responses, expression of the ionotropic Glutamate receptor GluR3 was assessed over time by RT-PCR of T-cell mRNA at various intervals following incubation with Glutamate. In addition, the effect of stimulation by Glutamate on the abundance of surface-GluR3 protein-positive T-cells cells was investigated.

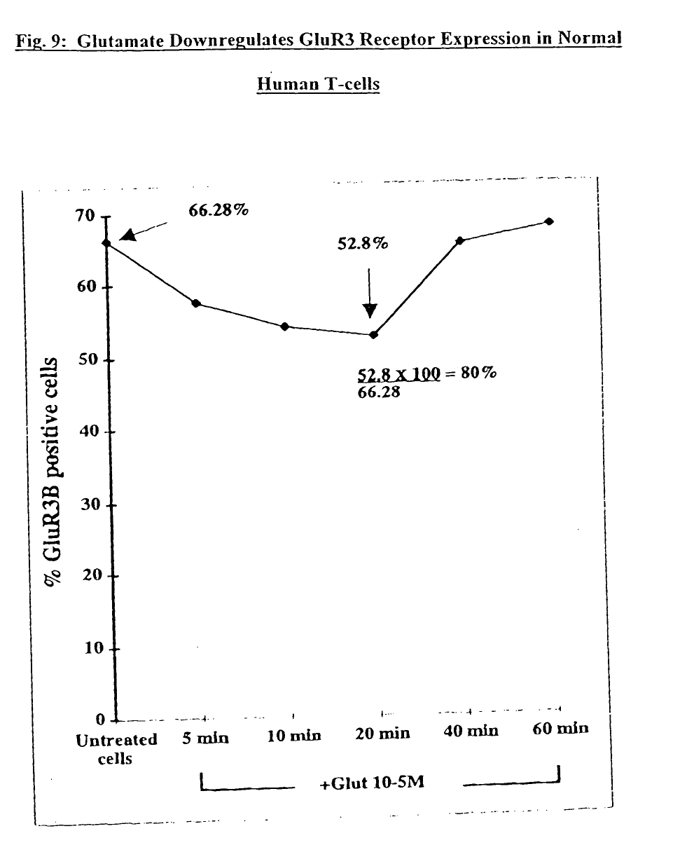

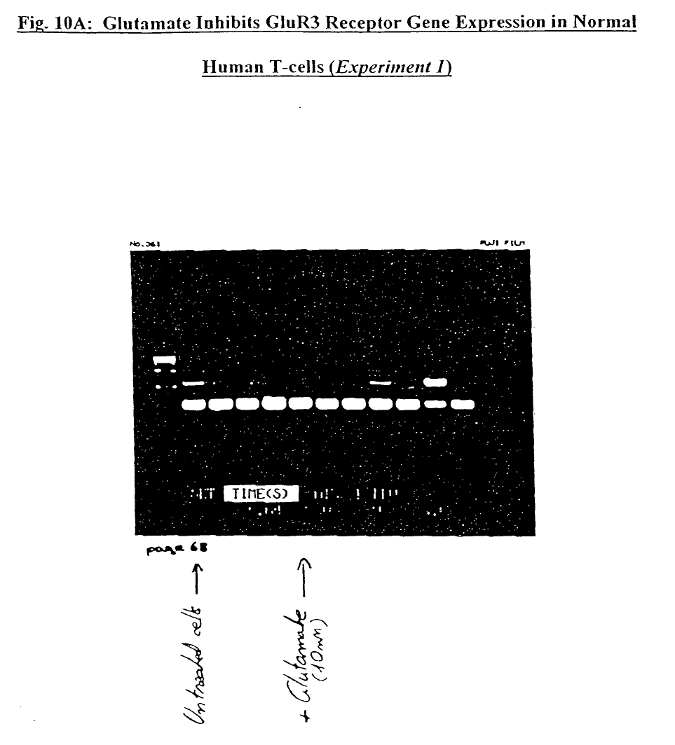

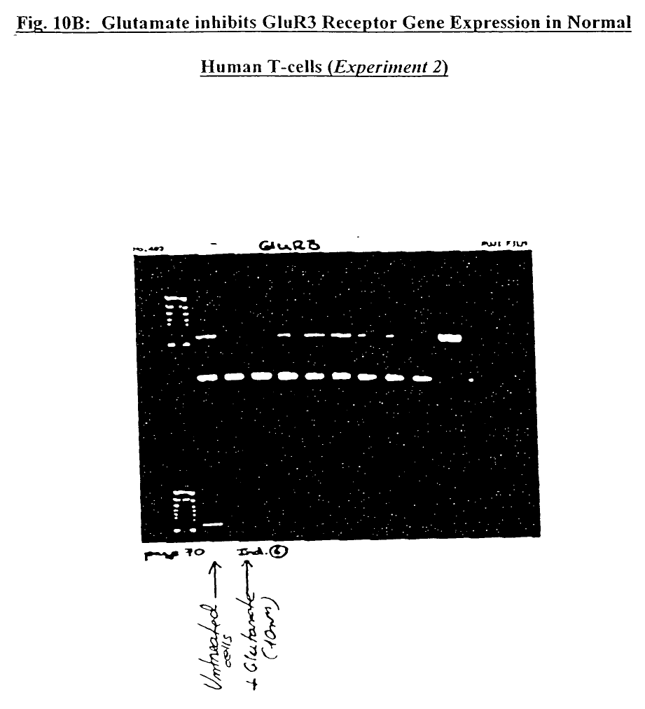

FIG. 9 demonstrates that 10⁻⁵ M Glutamate transiently down-regulates the expression of it's specific GluR3 receptor in normal human T-cells, to 80% of pre-Glutamate values, as detected by immunofluorescent labeling and FACS sorting. Double immunofluoresence staining, using a polyclonal rat anti-human GluR3 antibody confirmed the surface expression of the GluR3 receptor on human T-cells, and showed that its level decreases rapidly following treatment with Glutamate (FIG. 9, 0-20 min), the strongest inhibition occurring at 20 minutes incubation. A comparable reduction in the abundance of GluR3 mRNA following treatment of the normal human T-cells with 10 mM Glutamate was observed, as demonstrated by the RT-PCR amplification of GluR3 transcripts from treated and untreated cells (FIGS. 10A and 10B).

In addition, surface expression of GluR3 receptors was demonstrated, for the first time, in human T-cell leukemia (Jurkat) and mouse lymphoma (EL-4) cells, using the same anti-GluR3 antibodies (data not shown).

Taken together, these results indicate the existence of “cross-communication” between TCR and Glutamate receptors in normal and neoplastic human T-cells. Thus, a normal or elevated release of Glutamate in a variety of physiological and pathological conditions (specifically in neural tissue and brain extracellular fluid) in vivo can be directly “sensed” by patrolling T-cells via Glutamate receptors, with or without additional antigenic (TCR-mediated) stimulation, thus inducing and modulating specific T-cell functions.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and sequences identified by their genebank accession numbers mentioned in this specification are herein incorporated in their entirety by reference into the specification, to the same extent as if each individual publication, patent, patent application or sequence was specifically and individually indicated to be incorporated herein by reference. In addition, citation or identification of any reference in this application shall not be construed as an admission that such reference is available as prior art to the present invention.

1. An assay of determining an effect of Glutamate or a Glutamate analog on a T cell related disease or condition, the assay comprising:
exposing an organism having the T cell related disease or condition to at least one concentration of Glutamate or the Glutamate analog; and
assessing at least one T cell related symptom in said organism.

2. The assay of claim 1, wherein said Glutamate analog is selected from the group consisting of naturally occurring and synthetic analogs.

3. The assay of claim 1, wherein said Glutamate analog is a downregulator of T cell activation.

4. The assay of claim 1, wherein said Glutamate analog is an upregulator of T cell activation.

5. A method of modulating T cell activity, the method comprising exposing T cells to Glutamate or a T cell activity modulating Glutamate analog.

6. The method of claim 5, wherein exposing said T cells to said Glutamate or said T cell activity modulating Glutamate analog is performed in vitro.

7. The method of claim 5, wherein exposing said T cells to said Glutamate or said T cell activity modulating Glutamate analog is performed in vivo.

8. The method of claim 5, wherein said T cell activity modulating Glutamate analog is an upregulator, causing increased T cell activity.

9. The method of claim 5, wherein said Glutamate analog is selected from the group consisting of naturally occurring and synthetic analogs.

10. The method of claim 5, wherein said T cell activity modulating Glutamate analog is a downregulator, causing decreased T cell activity.

11. The method of claim 5, wherein said downregulator is a Glutamate receptor blocker.

12. A method of upregulating T cell activity in a mammalian subject, the method comprising administering to the subject a therapeutically effective amount of Glutamate or a T cell upregulating Glutamate analog, said amount being sufficient to upregulate T cell activity, thereby upregulating said T cell activity in the mammalian subject.

13. The method of claim 12, wherein said upregulating Glutamate analog is selected from the group consisting of naturally occurring and synthetic analogs.

14. The method of claim 12, wherein administering said therapeutically effective amount of Glutamate or a T-cell upregulating Glutamate analog is performed ex vivo.

15. The method of claim 12, wherein administering said therapeutically effective amount of Glutamate or a T-cell upregulating Glutamate analog is performed in vivo.

16. The method of claim 12, wherein said subject is suffering from a T cell related disease or condition selected from the group consisting of congenital immune deficiencies, acquired immune deficiencies, infection, neurological disease and injury, psychopathology and neoplastic disease.
